(19) United States
(12) Patent Application Publication
(10) Pub. No.: US 2002/0006629 A1
(43) Pub. Date: Jan. 17, 2002

(54) Affinity matrix bearing tumor-associated carbohydrate-or glycopeptide-based antigens and uses thereof

(76) Inventors: Samuel J. Danishefsky, Englewood, NJ (US); Kenneth O. Lloyd, Bronx, NY (US); Zhi-Guang Wang, Dresher, PA (US); Lawrence J. Williams, New York, NY (US)

Correspondence Address: Choate, Hall & Stewart, Exchange Place, 53 State Street, Boston, MA, 02109, US

(21) Appl. No.: 09/794,905

(22) Filed: Feb. 27, 2001

Related U.S. Application Data
(60) Provisional application No. 60/185,887, filed on Feb. 29, 2000.

(51) Int. Cl. G01N 33/574; A61K 39/395; C08B 37/00; C07K 14/435

(52) U.S. Cl. 435/7.23; 424/155.1; 530/395; 536/53

(57) ABSTRACT

An affinity matrix having a tumor-associated carbohydrate- or glycopeptide-based antigen bound to the matrix is provided. The affinity matrix is used to isolate, characterize, and quantitate functional antibodies or antigen-binding molecules to the tumor-associated carbohydrate- or glycopeptide-based antigen. The invention also provides a method of preparing the affinity matrix. In addition the invention provides for diagnostic and therapeutic uses of the isolated antibodies or antigen-binding molecules.

### PRIORITY INFORMATION

The present application claims priority under 35 U.S.C. § 119(e) to co-pending provisional patent application No. 60/185,887, filed Feb. 29, 2000, entitled “Affinity Matrix Bearing Tumor-Associated Carbohydrate- or Glycopeptide-Based Antigens for the Detection, Isolation, and Characterization of Antibodies and Antigen-Binding Molecules”, the entire contents of which are hereby incorporated by reference.

### GOVERNMENT SUPPORT

This invention was supported by funding from the National Institutes of Health (AI-16943, CA-28824, CA-71506 and CA-08748). Therefore, the government may have certain rights in the invention.

### BACKGROUND OF THE INVENTION

Oncogenesis is often associated with changes in the expression of cell surface carbohydrates. Among the many structural and functional transformations that attend oncogenesis, this altered expression of cell surface carbohydrates has recently emerged as a focal point for the development of vaccine strategies, Pardoll, D. M., Nature Med. 4,525-531 (1998); Ragupathi, G. & Livingston, P. O., Cancer Immunol. Immunother, 45,10-19 (1997). In some instances, the carbohydrate pattern displayed on the cell surface may be specific to the disease type. In others, the carbohydrate expression level may be markedly enhanced by the onset of disease. Many carbohydrates with potential clinical importance have been identified as either specific to the surface of a certain tumor cell or grossly over-expressed on the tumor cell surface, Lloyd, K. O, Am. J. Clin. Pathol. 87, 129-139 (1987): Hakomori, S., Cancer Res. 56, 5309-5318 (1996). Several of these tumor-associated carbohydrate- or glycopeptide-based antigens including globo-H, Lewis Y (“Le^{y}”), GM2, GD2, GD3, fucosyl GM1, S-Tn, Tn, TF and glycosylated segments of muc1 and muc 2, obtained by total synthesis and/or isolated from natural sources have been purified and conjugated to a protein carrier such as keyhole limpet hemacyanin (“KLH”) and administered with the immunologic adjuvant QS-21 as a carbohydrate-based cancer vaccine. See, for example, Livingston, P. O., Natoli, E. J., Calves, M. J., Socket, E., Oettgen, H. F., & Old, L. J., Proc. Natl. Acad. Sci. USA 84,2911-2915 (1987); Livingston, P. O., Wong, G. Y. C., Adler, S., Tao, Y., Padavan, M., Parente, R., Hanlon, C., Calves, M. J., Helling, F. Ritter, G., Octtgen, H. F. & Old, L. J., J Clin. Oncol. 12, 1036-1044 (1994); Ragupathi, G., Slovin, S. F., Adler, S., Sames, D., Kim, I. J., Kim, H. M., Spassova, M., Bommann, W. G., Lloyd, K. O., Scher, H. I., Livingston, P. O. & Danishefsky, S. J., Angew. Chem. Int. Ed. 38,563-566 (1999); Solvin, S. F., Ragupathi, G., Adler, S., Ungers, G., Terry, K., Kim, S., Spassova, M., Bornmann, W. G., Fazzari, M., Dantis, L., Olkiewicz, K., Lloyd, K. O., Livingston, P. O., Danishefsky, S. J. & Scher, H. I., Proc., Natl. Acad. Sci. USA 96,5710-5715 (1999); Kudryashov, V., Kim, H. M., Ragupatihi, G., Danisliefsky, S. J., Livingston, P. O. & Lloyd, K. O., Cancer Immunnol. Immunother, 45,281-286 (1998).

Conceptually, the goals of a tumor-associated carbohydrate or glycopeptide-based vaccine initiative are to educate the immune system to identify certain glyco-patterns as pathenogenic. Livingston, P. O., Zhang, S., and Lloyd, K. O., Cancer Immunol. Immunother. 45, 1-9 (1997). In this way an immune response is stimulated that is directed against cells bearing the tumor-associated carbohydrate or glycopeptide, and thus an effective policing mechanism against circulating cancer cells and micrometastases results. Once access to an antigen is achieved, and a viable vaccine is formulated, immunological characterization in animal models can be followed by clinical evaluation. In the best case, the immune response stimulated in humans would be subject to quantitative characterization. This in turn would provide a firm immunological base, as well as insights into therapeutic efficacy and identify potential modalities of vaccine optimization.

Significantly, in studies involving vaccination of mice with these carbohydrate-based vaccines, consistent induction of IgM and IgG antibodies reactive with tumor cells has been noted. Livingston, P. O., Wong, G. Y. C., Adler, S., Tao, Y., Padavan, M., Parente, R., Hanlon, C., Calves, M. J., Helling, F., Ritter, G., Oettgen, H. F. & Old, L. J., J. Clin. Oncol. 12, 1036-1044 (1994); Takahashi, J., Johnson T. D., Nishinaka Y., Morton, D. L., Irie, R. F., J. Invest. Dematol. 112, 205209 (1989). Furthermore, the vaccine-induced antibody response against GM2 has been associated with an improved disease-free survival.

One of the most promising candidates that has emerged is globo-H hexasaccharide, which has been synthesized, conjugated to KLH, and administered with the immunologic adjuvant QS-21 as a vaccine for patients with prostate cancer who have relapsed after primary therapies such as radiation or surgery. Ragupathi, G., Slovin, S. F., Adler, S., Sames, D., Kim, I. J., Kim, H. M., Spassova, M., Bornmann, W. G., Lloyd, K. O., Scher, H. I., Livingston, P. O. & Danishefsky, S. J., Angew. Chem. Int. Ed. 38, 563-566 (1999); Solvin, S. F., Ragupathi, G., Adler, S., Ungers, G., Terry, K., Kim, S., Spassova, M., Bornmann, W. G., Fazzari, M., Dantis, L., Olkiewicz, K., Lloyd, K. O., Livingston, P. O., Danishefsky, S. J. & Scher, H. I., Proc. Natl. Acad Sci. USA 96, 5710-5715 (1999). Globo-H(Fuc 1-2Gal,1-3GaINAc,1-3Gal 1-4Gal,) has been identified on human prostate, breast, and small cell lung carcinomas, as well as in a restricted number of normal epithelial tissues. Livingston, P. O., (1995) Cancer Biol. 6, 357-366; Zhang, S., Cordon-Cardo, C., Zhang, H. S., Reuter, V. E., Adler, S., Hamilton, W. B., Lloyd, K. O. & Livingston, P. O., Int. J. Cancer 73, 42-49 (1997). Globo-H was originally isolated as a ceramide-linked glycolipid by Hakamorl and co-workers, from the human breast cancer cell line MCF-7. Bremer, E. G., Levery, S. B., Sonnino, S., Ghidoni, R., Canevari, S., Kannagi, R. & Hakamor, S., J. Biol. Chem. 259, 14773-14777 (1984). It is expressed at the cancer cell surface as a glycolipid, and possibly as a glycoprotein. Globo-H has been further characterized by several methods, including immunohistochemistry using murine monoclonal antibody (mAb) MBr1. Zhang, S., Cardan-Cordo, C., Zhang, M. S., Reuter, V. E., Adluoi, S., Hamilton, N. R, Lloyd, K. O., and Livingston, P. O., Int. J. Cancer 73, 42-49 (1997). These studies have demonstrated the expression of cell surface carbohydrates, which were assumed to be globo-H, that react with the MBr1 antibody on many normal tissues, including normal breast, pancreas, small bowel, and prostate tissue. The antigen in these tissues is, however, predominantly localized at the secretory borders where access to the immune system is restricted. However, there is enhanced expression of MBr I-positive antigens on both primary and metastatic prostate cancer specimens. Zhang, S., Zhang, H. S., Reuter, V. E., Slovin, S. F., Scher, H. I. & Livingston P. O., Clin. Cancer Res. 4, 295-302 (1998). This enhanced expression of carbohydrate antigens, which are assumed to be globo-H, on both primary and metastatic prostate cancer specimens, provided the rationale for evaluating globo-H as a candidate target antigen in clinical trials for patients with relapsed prostate cancer. An estimated 37,000 men in the United States will be killed by prostate cancer in 1999. Prostate cancer is the most frequently diagnosed cancer to afflict nonsmoking men, and thus the need for effective therapies is in great demand. Indeed, one of the most difficult features of prostate cancer therapy is the limited options available to combat it. Aside from radical prostatectomy, hormonal chemotherapy, and radiation treatment, few regimens are available to achieve disease relief. Once the primary cancer has been treated, recurrence is often associated with an unfavorable outcome. Thus, the need for additional therapies to combat this disease is great. Further, the advantage of monitoring disease progression with prostrate serum antigen (“PSA”) makes prostate cancer an excellent candidate for a globo-H-based anti-cancer vaccine strategy.

In developing a globo-H based vaccine, a method of synthesis that provides suitable quantities of globo-H and related analogs for testing was developed and is described in U.S. Pat. No. 5,708,163 and in the pending divisional Ser. No. 08/977,215, and the pending C.I.P. Ser. No. 09/016,611. After synthesis of globo-H, a vaccine was formulated wherein the globo-H oligosaccharide was conjugated to keyhole limpet hemacyanin (KLH) and ultimately administered with the adjuvant QS-21, in mice in which carbohydrate immunogenicity was demonstrated. Park, T. K., Kim, I. J., Hu, S., Bilodeau, M. T., Randolph, J. T., Kwon, O. & Danishefsky, S. J., J Am. Chem. Soc. 118, 11488-1500 (1996). Moreover, initial clinical evaluation in relapsed prostate cancer patients demonstrated the successful and safe induction of antibodies specifically focused against globo-H, i.e., tolerance was broken but no autoimmune reactions occurred. Ragupathi, G., Slovin, S. F., Adler, S., Sames, D., Kim, I. J., Kin, H. M., Spassova, M., Bormann, W. G., Lloyd, K. O., Scher, H. I., Livingston, P. O. & Danishefsky, S. J., Angew. Chem. Int. Ed. 38, 563-566 (1999); Solvin, S. F., Ragupathi, G., Adler, S., Ungers, G., Terry, K., Kim, S., Spassova, M., Bornmann, W. G., Fazzari, M., Dantis, L., Olkiewicz, K., Lloyd, K. O., Livingston, P. O., Danishefsky, S. J. & Scher, H. I., Proc. Natl. Acad. Sci. USA 96, 5710-5715 (1999).

Significantly, there is a strong formal analogy between cancer patients suffering from recurrence and individuals re-exposed to infectious disease. In both cases, the primary antigenic targets are localized and circulating microscopic “pathogens.” In the context of immune stimulation with a carbohydrate-based anti-cancer vaccine the similarity is even more evident. As carbohydrates are considered to be killer T-cell independent antigens (and thus not expected to induce a cytotoxic T-cell response), any positive clinical outcome would most likely result from antibody-mediated effector mechanisms such as complement-dependent cytotoxicity (“CDC”), antibody-dependent cellular cytotoxicity (“ADCC”), and induction of inflammation. Thus, the mechanism of antibody effect against cancer cells appear to be similar to the action of antibodies against bacteria, which also involve predominantly CDC, ADCC, and induction of inflammation. In both cases immunity consists of antibodies serving as the primary mechanism to limit spread of disease.

Clearly, demonstrable immunogenicity in preclinical models is an important litmus test before continuing on to the clinical setting. In such cases, vaccination of animals has supported the mechanistic analogy described previously. For example, CDC against tumors bearing the globo-H epitope for mice vaccinated with a globo-H-KLH construct have been reported. Ragupati, G., Slovin, S. F., Adler, S. Sames, D., Kim, I. J., Kim, H. M., Spassova, M., Bornmann, W. G., Lloyd, K. O., Scher, H. I., Livingston, P. O. & Danishefsky, S. J., Angew. Chem. Int. Ed. 38, 563-566 (1999). Furthermore, in models where GM2 was the target antigen, both passively administered monoclonal antibodies and vaccine-induced polygonal antibodies were able to defeat establishment of subsequently administered tumor cells and to eliminate related micrometastases. These results have led to certain cautious optimism, suggesting that a sufficiently potent antibody response could, in principle, eliminate circulating cancer cells and micrometastases in cancer patients.

There are, however, two critical interrelated issues to be addressed in progressing from a murine to a human vaccination setting. Most carbohydrate antigens evaluated for immunogenicity in mice are not endogenous to them. Hence, it is not remarkable that an immune response would be mounted against such a foreign carbohydrate entity. The situation is quite different in humans. Though often pathological and aberrantly expressed, tumor-associated carbohydrates are not foreign to the human subject; therefore, tolerance must be broken for an immune response to be registered. Importantly, the caveat exists that the response must not lead to auto-immunity, due to the expression of many carbohydrate antigens on normal tissue. While vaccines based on globo-H, LewisY, Tn and TF are still progressing through various stages of clinical evaluation, vaccine-induced antibodies against GM2 have correlated with improved disease-free and overall survival in melanoma patients. Livingston, P. O., Wong G. Y. C., Adler, S., Tao, Y. Padavan, M., Parente, R., Hanlon, C., Calves, M. J., Helling, F., Ritter, G., Oettgen, H. F. & Old, L. J., J Clin. Oncol. 12,1036-1044 (1994); Livingston, P. O., Zhang, S. & Lloyd, K. O., Cancer Immunol. Immunother., 45, 1-9 (1997). As in the case of globo-H, these vaccine-induced antibodies were able to mediate CDC.

As discussed in detail above, through clinical trials on the tumor-associated carbohydrate-based or glycopeptide-based antigen, the mechanism of antibody action against cells expressing tumor-associated carbohydrate-based or glycopeptide-based antigens has been demonstrated to include CDC. Ragupathi et al. Angew. Chem. Int. Ed. 38, 563-566 (1999). Although the globo-H vaccine and other tumor-associated carbohydrate-based vaccines have been shown to induce antibody responses, there has been no quantitative data on the antibody levels achieved in immunized patients by these or other anti-cancer vaccines. Clearly, it would be useful to more completely identify and quantitate antibodies induced by the administration of anti-cancer vaccines as discussed above. There also remains a need to further identify and quantitate antibodies and antigen-binding molecules, not only for the development of these previously mentioned carbohydrate- and glycopeptide-based vaccines, but also for novel tumor-associated carbohydrate- and glycopeptide-based analogues that are being developed through the efforts of synthetic chemistry.

### SUMMARY OF THE INVENTION

In recognition of the need for the quantification, characterization, and isolation of antibodies generated by the administration of carbohydrate- and glycopeptide-based antigens and conjugates thereof, the present invention provides an affinity matrix comprising tumor-associated carbohydrate- or glycopeptide-based antigen bound to the matrix. In certain embodiments, the matrix comprises a solid support, including, but not limited to, tentagel, agarose, acrylic or polyacrylamide. In certain embodiments, the solid support comprises agarose. In certain other embodiments, the antigen utilized in the affinity matrix comprises monomeric or clustered globo-H-oligosaccharide, Lewis Y oligosaccharide, GM2, GD2, GD3, fucosyl GM1, S-Tn, Tn, TF, KH-1, N3, glycosolated segments of muc 1 or muc 2, or combinations thereof.

In other embodiments of the invention, an affinity matrix comprising synthetic tumor-associated carbohydrate- or glycopeptide-based antigen bound to the matrix is provided. In certain embodiments, the matrix comprises a solid support, including, but not limited to, tentagel, agarose, acrylic or polyacrylamide. In certain embodiments, the solid support comprises agarose. In certain other embodiments, the synthetic antigen utilized in the affinity matrix comprises synthetic monomeric or clustered globo-H-oligosaccharide, Lewis Y oligosaccharide, GM2, GD2, GD3, fucosyl GM1, S-Tn, Tn, TF, KH-1, N3, glycosolated segments of muc 1 or muc 2, or combinations thereof.

In another embodiment of the invention, an affinity matrix is provided comprising monomeric or clustered globo-H-oligosaccharide bound to an agarose support. In yet other embodiments, an affinity matrix is provided comprising monomeric or clustered Lewis Y oligosaccharide bound to an agarose support. In still other embodiments, an affinity matrix comprising monomeric or clustered Tn bound to an agarose support is provided. In yet other embodiments, an affinity matrix comprising monomeric or clustered TF bound to an agarose support is provided.

In another aspect of the invention, a method for preparing an affinity matrix is provided comprising the steps of 1) providing monomeric or clustered tumor-associated carbohydrate- or glycopeptide-based antigen, or a combination thereof; and 2) contacting said carbohydrate- or glycopeptide-based antigen with a solid support, whereby the step of contacting effects binding of the antigen to the support. In certain embodiments, the step of providing monomeric or clustered carbohydrate- or glycopeptide-based antigen comprises providing synthetic monomeric or clustered carbohydrate- or glycopeptide-based antigen. In yet other embodiments, the step of providing monomeric or clustered carbohydrate- or glycopeptide-based antigen comprises providing synthetic monomeric or clustered carbohydrate- or glycopeptide-based antigen having terminal allyl functionality. In still other embodiments, the step of providing further comprises converting the terminal allyl functionality to a corresponding in situ aldehyde. In certain other embodiments, the step of providing monomeric or clustered carbohydrate- or glycopeptide-based antigen comprises providing synthetic monomeric or clustered carbohydrate- or glycopeptide-based antigen having a terminal amino, thio or acid functionality. It will also be appreciated that, in other embodiments, the method of the present invention, after the step of contacting, further comprises the steps of 1) capping any residual functionality present on the solid support; and 2) treating the affinity matrix with a suitable reagent to remove protecting groups present in the support-bound carbohydrate- or glycopeptide-based antigen. In certain embodiments, globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof is used in the method of the invention; and in certain other embodiments, monomeric or clustered synthetic globo-H oligosaccharide, synthetic Lewis-Y oligosaccharide, synthetic Tn, or synthetic TF is utilized.

In yet another aspect of the present invention, a method for isolating antibodies or antigen-binding molecules is provided comprising the steps of 1) providing a solution comprising antibodies or antigen-binding molecules; 2) contacting the solution with an affinity matrix, which affinity matrix comprises carbohydrate- or glycopepetide-based antigens that are capable of binding to said antibodies or antigen-binding molecules; and 3) eluting the antibodies or antigen-binding molecules from the affinity matrix. In certain embodiments, the method further includes an additional step, after the step of contacting, of washing the affinity matrix to remove unbound substrates. In certain other embodiments, the step of providing a solution comprises providing blood fluids from a subject, and in certain embodiments, these blood fluids are provided after the subject has been immunized with a monomeric or clustered tumor associated carbohydrate- or glycopeptide based antigen. It will be appreciated that the present invention also encompasses additional steps for quantifying or characterizing the isolated antibodies or antigen-binding molecules. Additionally, in certain embodiments, the antigen comprises monomeric or clustered globo-H-oligosaccharide, Lewis Y oligosaccharide, GM2, GD2, GD3, fucosyl GM1, S-Tn, Tn, TF, KH-1, N3, glycosolated segments of muc 1 and muc 2, or combinations thereof, and in certain other embodiments comprises monomeric or clustered globo-H oligosaccharide, Lewis Y oligosaccharide, Tn, TF, or a combination thereof. It will also be appreciated that in certain embodiments, the antibodies or antigen-binding molecules isolated retain their functionality.

In yet another aspect, the present invention provides a method of detecting a cancer in a subject comprising the steps of 1) providing a solution comprising blood fluids from a subject; 2) contacting the solution with an affinity matrix, wherein said affinity matrix comprises tumor-associated carbohydrate- or glycopeptide-based antigens bound to the matrix; 3) treating the affinity matrix with a reagent suitable to elute antibodies or antigen-binding molecules bound to the tumor-associated carbohydrate- or glycopeptide-based antigens present in the affinity matrix; and 4) determining the presence of antibodies or antigen-binding molecules. In certain embodiments, the tumor-associated carbohydrate- or glycopeptide-based antigen comprises monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof. In certain other embodiments, the step of providing blood fluids from a subject comprises immunizing a subject with a monomeric or clustered tumor-associated carbohydrate- or glycopeptide-based antigen and collecting a blood sample from the subject. In still other embodiments, the method further comprises repeating steps (a)-(d) at one or more specific time intervals to monitor the progress of treatment over a specific period of time of a type of cancer having a tumor-associated carbohydrate- or glycopeptide-based antigen associated therewith.

In still another aspect of the present invention, a method of treating cancer in a subject is provided comprising the steps of 1) isolating antibodies or antigen-binding molecules, wherein the step of isolating comprises providing a solution comprising blood fluids from a subject; contacting the solution with an affinity matrix, whereby said affinity matrix comprises tumor-associated carbohydrate- or glycopeptide-based antigens; and treating the affinity matrix with a reagent suitable to elute antibodies or antigen-binding molecules bound to the tumor-associated carbohydrate- or glycopeptide-based antigens present in the affinity matrix; 2) conjugating one or more therapeutic agents to the isolated antibodies or antigen-binding molecules; and 3) re-administering the conjugated antibodies or antigen-binding molecules to the subject. In certain embodiments, the cancer to be treated is prostrate, breast, colon, ovarian, pancreatic, melanoma, neuroblastoma, or small cell lung cancer. In certain other embodiments, the one or more therapeutic agents are radioactive isotopes or anti-cancer agents. In certain other embodiments the antibodies isolated are antibodies capable of binding to monomeric or clustered globo-H-oligosaccharide, Lewis Y oligosaccharide, GM2, GD2, GD3, fucosyl GM1, S-Tn, Tn, TF, KH-1, N3, glycosolated segments of muc 1 and muc 2, or combinations thereof. In certain other embodiments, the antibodies are capable of binding to monomeric or clustered globo-H antigen, LewisY antigen, Tn antigen, TF antigen, or a combination thereof. In still other embodiments, said antibodies are induced by a monomeric or clustered Lewis Y vaccine, Globo-H vaccine or Tn vaccine, TF vaccine, or a combination thereof.

In still another aspect of the present invention, method of imaging cancer metastases in a subject is provided comprising the steps of: 1) isolating antibodies or antigen-binding molecules, wherein the step of isolating comprises: providing a solution comprising blood fluids from a subject; contacting the solution with an affinity matrix, whereby said affinity matrix comprises tumor-associated carbohydrate- or glycopeptide-based antigen; and treating the affinity matrix with a reagent suitable to elute antibodies or antigen-binding molecules bound to the tumor-associated carbohydrate- or glycopeptide-based antigens present in the affinity matrix; 2) labeling the isolated antibodies or antigen-binding molecules with imaging agents; and 3) re-administering the labeled antibodies or antigen-binding molecules to the subject. In certain embodiments, the imaging substance is a radioactive isotope. In still other embodiments, the isolated antibodies are antibodies capable of binding to monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof. In yet other embodiments, the antibodies are capable of binding to monomeric or clustered globo-H antigen, Lewis-Y antigen, Tn antigen, TF antigen, or a combination thereof.

### DESCRIPTION OF THE DRAWING

FIG. 1A depicts the results of affinity chromatography using a globo-H-agarose column on the sera of a patient (No. 4) following vaccination of the subject with a globo-H vaccine.

FIG. 1B depicts the results of affinity chromatography of the same patient prior to vaccination with the vaccine. Fractions are assayed for protein levels (OD280: -▪-) and reactivity with globo-H-ceramide by ELISA(-Δ-). The initial wash buffer is PBS. The second wash (arrow 1) is with 1 M HCl. The final elution buffer (arrow 2) is 0.05M glycine-HCl at pH 2.5.

FIG. 2 depicts the SDS-PAGE analysis of fractions isolated by affinity chromatography on a globo-H-agarose column from the sera of six patients immunized with globo-H-KLH. Lanes 1-6 are low pH-eluted fractions from patients Nos.1 to 6. Lane 7 is the eluted fraction from patient No. 3 further purified to remove HSA. Lane S is protein standards (220, 130, 90, 70, 60, 40, 30, and 20 kDa). The migration rates of IgM, IgG, and HSA are indicated. The samples are separated on a 7% polyacrylamide gel under non-reducing conditions and then stained with Coomassie Blue.

FIG. 3 depicts the results of the analysis of specificity of antibody fractions isolated from five immunized patients: Panel A—patient No. 1; Panel B—patient No. 2; Panel C—patient No. 3; Panel D—patient No. 4; and Panel E—patient No. 5). Panel F is the results of anti-globo H monoclonal antibody VK-9. Reactivity is measured with a direct ELISA using rabbit anti-human IgG (H and L)-alkaline phosphatase as the second antibody. The test compounds are as follows:

Lane 1: globo H-Cer

Lane 2: galactosyl-globoside-Cer (SSEA-3 antigen)

Lane 3: globoside-Cer

Lane 4: Le^{y}-(Fuc 1-2Gal 1-4[Fuc 1-3]GlcNAc, 1-3Gal)-BSA

Lane 5: Le^{b}-(Fuc 1-2Gal 1-3[Fuc 1-4]GlcNAc, 1-3Gal)-BSA

Lane 6: Le^{x}-(Gal,1-4[Fuc 1-3]GIcNAc)-PAA

Lane 7: Le^{x}-Cer

Lane 8: Le^{a}-(Gal, 1-3[Fuc 1-4]GIcNAc)-PAA

Lane 9: H type 2(Fuc 1-2Gal,1-4G1cNAc)-PE

Lane 10: H type 2-PAA

Lane 11: H type1(Fuc 1-2Gal,1-3GlcNAc,1-3Gal)-BSA

Lane 12: Le^{a}-PAA

Lane 13: Lactose-CETE

Lane 14: Gal 1-4Gal, 1-4Glc-CETE

Lane 15: Gal 1-4GIcNAc-CETE-B SA

Abbreviations: Cer=ceramide; BSA=bovine serum albumin; PAA=polyacrylamide; PE=phosphatidyl ethanolamine; CETE=2-(carbomethoxyethylthio) ethyl.

FIG. 4 depicts the yields for immunoglobulins isolated from the sera of six patients who had been immunized with a globo-H-KLH vaccine and from the sera of three normal individuals. The sera was analyzed for protein content using the Lowry assay. The immunoglobulins are isolated by affinity chromatography on a globo-H agarose column.

FIG. 5 depicts a chart of the purified antibodies from patients' sera. This chart shows the composition of antibodies produced by the patients immunized with the globo-H vaccine. The chart reveals that IgM and IgG antibodies are produced.

FIG. 6 depicts a diagram of the production of the KLH vaccine and affinity column for globo-H and Le^{y}. This figure shows the conversion of protected polysaccharides globo-H hexasaccharide and Le^{y} pentasaccharide to functional affinity matrices: globo-H bonded agarose and Le^{y} bonded agarose.

FIG. 7 depicts a diagram of the synthesis of the globo-H hexasaccharide and the Le^{y} pentasaccharide having terminal allyl groups.

FIG. 8 depicts certain inventive tumor-associated glycopeptide-based antigens for use in the present invention.

### DETAILED DESCRIPTION OF CERTAIN EMBODIMENTS OF THE INVENTION

As discussed above, although carbohydrate antigens have been shown to induce antibody responses and have been useful in the development of cancer vaccines, there remains a need to better evaluate the level and specificity of vaccine induced antibodies and to accomplish enrichment and separation of these antibodies elicited through vaccination. In recognition of this need, the present invention provides affinity matrices comprising carbohydrate- or glycopeptide-based antigens bound thereto, and methods for the preparation thereof.

Specifically, the inventive affinity matrix represents the first use of affinity chromatography in conjunction with a total synthesis driven tumor-associated carbohydrate- or glyocpeptide-based vaccination strategy. The present invention addresses the need to have access to sizable quantities of human antibodies specific to tumor-associated carbohydrate- or glycopeptide-based antigens, which allows for mechanistic evaluation of antibody production as well as diagnostic and therapeutic applications. The application of affinity chromatography to enhance the insight into the immunological factors of vaccination is valuable in the clinical evaluation of cancer patients.

The affinity matrix of the present invention enables the quantitative analysis of the antibody immune response of cancer patients raised against a tumor-associated carbohydrate- or glycopeptide-based vaccine, i.e. the immune response of prostrate cancer patients raised against the globo-H-KLH vaccine. The purity of the antibodies obtained by the affinity matrix of the present invention allows detailed characterization and avoids uncertainties inherent to serological assays. The data thus obtained provides a reliable and conclusive assessment of the stimulated immune response. Such affinity matrices are invaluable tools for immunological investigations and in diagnostic and therapeutic applications. For example, a library of polyclonal antibody isolation tools provides a straightforward and reliable avenue by which to evaluate vaccine immune response in cancer patients, such that even polyvalent vaccination strategies are quantitatively assessed with ease.

Affinity Matrices and Preparation Thereof

According to the method of the present invention, an affinity matrix comprising carbohydrate- or glycopeptide-based antigen bound to a solid support is provided, which method comprises providing carbohydrate- or glycopeptide-based antigen; and contacting the carbohydrate- or glycopeptide-based antigen with a solid support, whereby the step of contacting the carbohydrate- or glycopeptide-based antigen with a solid support effects binding of the antigen to the support.

In certain embodiments, the present invention provides affinity matrices whereby the carbohydrates or glycopeptides of interest are generally tumor-associated carbohydrate or glycopeptide-based antigens. As used herein, the term “tumor-associated carbohydrate or glycopeptide-based antigens” is intended to encompass those antigenic structures that are functionally and/or structurally equivalent to those found on the surfaces of cancer cells. For example, certain carbohydrate- and glycopeptide-based antigens used in the present invention are both structurally and functionally the same as those found on the surfaces of tumor cells (e.g., Globo-H, Le^{y}, KH-1, Fucosyl-GM1, to name a few). In certain other embodiments, carbohydrate or glycopeptide-based antigens used are based upon the structures of certain antigens found on tumor cells (e.g., Globo-H), but may represent truncated or elongated versions of these antigens, or may additionally represent isomeric versions of these antigens. In certain other embodiments, other analogues of certain carbohydrate- or glycopeptide-based antigens, or alternate carbohydrate or glycopeptide structures are provided. It will be appreciated that these structures may be functionally equivalent (for example, the antigen is capable of inducing antibodies that interact with antigens found on the surfaces of tumor cells, and thus are also therapeutically useful), and thus are also useful for the preparation of the inventive affinity matrices.

It will also be appreciated that the antigens for use in matrices of the present invention can be provided in monomeric or in clustered form. The term “clustered” as used herein is intended to incorporate those structures having more than one carbohydrate antigen attached to a peptide backbone. For example, in certain embodiments, clustered antigens are provided, whereby the same type of antigen (e.g., Globo-H) is attached to the peptidic backbone. In certain other embodiments, clustered antigens are provided, whereby more than one type of carbohydrate antigen is attached to the peptidic backbone.

In certain embodiments, as described above and herein, the carbohydrate antigens include, but are not limited to, isolated or synthetic monomeric and/or clustered globo-H oligosaccharide, Le^{y} oligosaccharide, GM2, GD2, GD3, fucosyl GM1, S-Tn, Tn, TF, KH-1, N3, or glycosylated segments of muc 1 and muc 2, and truncated, elongated, or isomeric versions thereof. For a discussion of the synthesis of several of these structures, see Danishefsky et al. Angew. Chem. Int. Ed. Engl. 2000, 39, 836-863, the entire contents of which are hereby incorporated by reference. Additionally, as detailed above, the tumor-associated carbohydrate- or glycopeptide-based antigens can be synthesized as monomeric and/or clustered moieties and then converted into functional affinity matrices. The synthesis of these oligosaccharides and glycopeptides is disclosed in U.S. Pat. No. 5,708,163 and in the pending applications Ser. Nos. 08/977,215; 09/016,611; 08/506,251; 09/194,795; 09/043,713; 09/276,595; 09/480,280; 09/083,776; 09/276,595; and 09/641,742, which are herein incorporated by reference in their entirety.

It will be appreciated that once desired carbohydrate- or glycopeptide-based antigenic structures are selected, the antigen can be bound to a matrix in a variety of ways. In certain embodiments, the affinity matrix is a solid support. Suitable solid supports include, but are not limited to, tentagel, sepharose, agarose, acrylic, and polyacrylamide. In certain embodiments, the solid support is preferably agarose, due to its ease of preparation and analysis, and as well as performance. It will be appreciated that the solid support is also suitably functionalized for reaction with the antigenic structures as described herein. In certain embodiments, the solid support utilized is amine functionalized, or is functionalized with MBS (m-malemidobenzoyl N-hydroxysuccinimide).

According to the method of the present invention, a variety of linkages can be utilized to effect attachment of antigens to the matrix. For example, in certain embodiments, amide formation is effected (A. Williams & I. A. Ibrahim, J. Amer. Chm. Soc., 103:70790-7095 (1981)). In certain other embodiments, organomercurial addition (D. Tsuru, K, Fujiwara, & K. Kado, J. Biochem, 84:467-476 (1978)) is utilized, and in still other embodiments reductive amination (M. A. Bemskin & L. D. Hall, Carbohydr. Res. 78:C1 (1980)), is employed in the method of the present invention. Additionally, as also demonstrated herein, a maleimide coupling reaction can be effected between MBS functionalized solid support and thiol containing antigens.

As detailed above, the solid support is selected and suitably functionalized so that it is capable of reaction with a suitably functionalized tumor-associated carbohydrate-based antigen to effect attachment of the antigen to the affinity matrix. In certain embodiments, particularly for reaction with amine functionalized solid support, tumor-associated carbohydrate- or glycopeptide-based antigen is synthesized to have a terminal allyl, group, and then is converted to the reactive aldehyde in situ. Certain exemplary terminal allyl groups include, but are not limited to hexenyl, pentyl, butenyl, and allyl. In certain other embodiments, (for example for reaction with MBS functionalized solid support) tumor-associated carbohydrate- or glycopeptide-based antigen is synthesized to have a terminal amino, thio or acid group. It will also be appreciated that any amino, thio or acid group can be used, including, but not limited to, SH, NH₂, and COOH. As but one example, the synthesis of an oligosaccharide (a carbohydrate-associated tumor antigen) with an allyl group is disclosed in U.S. Pat. No, 5,708,163 “Synthesis of the Breast Tumor-Associated Antigen Defined by Monoclonal Antibody Mbrl and uses Thereof,” and in the pending divisional application, U.S. Ser. No. 08/977,215.

In certain other embodiments of the present invention, it is also useful to cap any residual functionality present on the solid support. In one exemplary embodiments, the residual amine functionality is capped using methods known in the art, such as, but not limited to, treatment with acetic anhydride. In still other embodiments of the present invention, it is also desirable to deprotect the attached tumor-associated carbohydrate- or glycopeptide-based antigen. The synthesized tumor-associated carbohydrate-or glycopeptide-based antigen-linked agarose is deprotected using procedures known in the art, including, but not limited to, treatment with Et₃N/MeOH/H₂O, NaOMe/MeOH, K₂CO₃/MeOH, MeOH/Zn(OAc)₂, or NaOH/H₂O.

As described above, the synthesized oligosaccharide or glycopeptide is generally a tumor-associated carbohydrate-or glycopeptide-based antigen, including, but not limited to, globo-H-oligosaccharide, Lewis Y oligosaccharide, or GM2, GD3, fucosyl GM1, or S-Tn, Tn, TF, KH-1, N3, glycosylated segments of muc 1 and muc 2, or combinations thereof. This process of synthesizing or preparing the oligosaccharide or glycopeptide and attaching it to an affinity matrix allows the affinity matrix to bear a tumor-associated carbohydrate- or glycopeptide-based entity found on the surface of cancer cells, including, but not limited to, globo-H-oligosaccharide, Lewis Y oligosaccharide, or GM2, GD3, fucosyl GM1, or S-Tn, Tn, TF, KH-1, N3, glycosolated segments of muc 1 and muc 2, or combinations thereof.

In one exemplary embodiment of the present invention (see Example 1), the method of the present invention can be utilized to attach Globo-H hexasaccharide to a solid support, by utilizing Globo-H having a terminal allyl moiety. Park, T. K., Kim, I. J., Hu, S., Bilodeau, M. T., Randolph, J. T., Kwon, O. & Danishefsky, S. J., J. Am. Chem. Soc. 118, 11488-11500 (1996). (See FIG. 7). For example, the allyl group is linked to the amine functionalized agarose by ozonolysis and reductive amination, followed by capping of residual amine functionality to give globo-H bound agarose. On-resin deprotection provides a fully functional globo-H antigen-bound affinity matrix.

In yet another exemplary embodiment (see also Example 2), in much the same way, the Le^{y} pentasaccharide, also as the allyl glycoside, (Danishefsky, S. J., Beher, V., Randolf, J. T., Lloyd, K. O., J. Am. Chem. Soc., 117,5701 and 5711 (1995)) is converted to the antigen specific affinity matrix. For comparison, the unprotected globo-H allyl glycoside is ozonolized and reductively coupled to amine functionalized agarose to give affinity material that is identical in all respects to globo-H bound agarose. Column material, for both globo-H and Le^{y}, is routinely produced on a 10 mL scale beginning with approximately 50 mg of protected carbohydrate.

In still other embodiments of the invention, other carbohydrate- and glycopeptide-based antigens can be prepared and utilized for the preparation of inventive affinity matrices. For example, KH-1 antigenic structures containing a terminal allyl functionality can be prepared as described in pending patent application Ser. No. 09/042,280 and can be subjected to ozonolysis to produce the aldehyde which can subsequently be attached to the solid support using methods described herein. In yet another example, Tn clusters can be prepared according to the methods described herein (see Examples 3 and 4), and can be attached to a solid support via maleimide coupling. In still other examples, glycopeptide-based clusters can be prepared according to the methods described in pending patent applications Ser. Nos. 09/083,776, 09/276,595, and 09/641,742, the entire contents of which are hereby incorporated by reference, which contain suitable terminal reactive moieties, that can be modified to effect attachment to the solid support, as discussed in more detail herein, to generate the inventive affinity matrices.

It will be appreciated that the examples as described above and herein are not intended to limit the scope of the present invention; rather, as discussed above, the inventive affinity matrices are intended to encompass the full scope of antigenic structures that are functionally and/or structurally equivalent to those found on the surfaces of cancer cells.

Uses of the Inventive Affinity Matrices

As described above, the inventive affinity matrices are useful in a variety of therapeutic contexts. For example, it would be useful to isolate functional antibodies or antigen-binding molecules so that these antibodies and antigen-binding molecules could then be used in therapeutic and diagnostic arenas.

Thus, in another aspect, the present invention provides a method for isolating antibodies or antigen-binding molecules comprising 1) providing a solution containing antibodies or antigen-binding molecules; 2) contacting the solution with an affinity matrix, which affinity matrix comprises carbohydrate- or glycocpeptide-based antigens that are capable of interacting with the antibodies of antigen-binding moleucles, as described in more detail above, and 3) eluting the antibodies or antigen-binding molecules from the affinity matrix. It will be appreciated that in certain embodiments, the antibodies or antigen-binding molecules isolated retain their functionality. In still other embodiments, the solution provided comprises a subject's blood fluids, and in some embodiments, the blood fluids are provided after the subject has been immunized with a carbohydrate- or glycopeptide-based antigen. In yet other embodiments, the method of the present invention further comprises a step of washing the affinity matrix to remove unbound substrates. In still other embodiments, the method further comprises quantifying or characterizing the isolated antibodies or antigen-binding molecules. As detailed above, the inventive affinity matrices can be constructed with a variety of tumor-associated carbohydrate- or glycopeptide-based antigens, and thus the method of the present invention encompasses the isolation of any antibodies or antigen-binding molecules that specifically interact with these tumor-associated carbohydrate- or glycopeptide-based antigens.

For example, according to the method of the present invention, a solution containing the antibodies or antigen binding molecules is applied to an affinity matrix having a tumor-associated carbohydrate-or glycopeptide-based antigen (monomeric and/or clustered form) bound to a matrix. The matrix is washed with a suitable solution such as PBS to effect removal of unbound substrates, such as other serum immunoglobulins and other serum proteins. These non-binding proteins flow freely through the matrix while the tightly bound tumor-associated carbohydrate- or glycopeptide based antibodies or antigen-binding molecules remain attached until the elution stage. These antibodies or antigen-binding molecules are then released with a mild release agent, such as glycine hydrochloride, and can be monitored spectrophotometrically. For example, as described herein, monoclonal mouse antibody (mAbVK9) is evaluated and, as expected, binds tightly to the column. Importantly, the antibody, once released, retains its full binding potency in subsequent ELISA analysis. Thus, the present invention also provides a method of isolation that allows the antibodies or antigen-binding molecules to retain their functionality after isolation. These functional antibodies or antigen-binding molecules are used in therapeutic or diagnostic applications.

In certain embodiments of the invention, as described in more detail herein, anti-globo-H antibodies, for example, are isolated from a subject's blood fluids. The antibodies are efficiently separated from other serological constituents. The isolated antibodies are readily quantified and their specificities are analyzed. Since no comparable data are available on antibodies resulting from the vaccination of other cancer patients, the observed levels are compared with those quoted in studies with bacterial polysaccharide vaccines that have been quantified. Remarkably, cancer patients immunized with a globo-H-KLH conjugate vaccine produce anti-globo-H antibody levels often exceeding those formed by immunization with bacterial polysaccharides. In addition, substantial quantities of both IgG and IgM antibodies are elicited, clearly indicating a class switch to IgG. The antibody reactivity profiles and subclass populations are also assessed. (See FIG. 5).

The present invention is also directed to the quantification and characterization of the isolated antibodies or antigen-binding molecules. After using the affinity matrix of the present invention to isolate the antibodies or antigen-binding molecules, the isolated antibodies are later quantified and characterized by methods commonly known in the art. See Examples 8 and 9. The present invention makes possible these analyses, which taken together, serve to clarify several aspects of the immune response and give several new insights to the carbohydrate-or glycopeptide-based vaccination strategy.

It will be appreciated that the ability to isolate antibodies or antigen-binding molecules is useful not only in the context of gaining new insights to carbohydrate- or glycopeptide-based vaccination strategy, but is also useful in therapeutic and other diagnostic contexts. For example, conjugation to other therapeutic or diagnostic agents can be effected, which permits treatment of a subject having cancer or permits the monitoring of a subject having cancer, or the antibodies and antigen-binding molecules can be analyzed and it can be determined whether a subject has cancer.

For example, isolated functional antibodies or antigen-binding molecules to the tumor-associated carbohydrate- or gylcopeptide-based based antigens, such as antibodies or antigen-binding molecules capable of interacting with globo-H (either natural antibodies or antibodies induced by a tumor-associated carbohydrate- or glycopeptide-based vaccine, such as the globo-H vaccine) or other carbohydrate or glycopeptide-based antigens (such as Lewis Y, fucosyl GM1, GM2, GD2, GD3, Tn, S-Tn, TF, KH-1, N3, glycosylated segments of muc 1 and muc 2, or combinations thereof)(naturally occurring or induced by vaccine), can then be conjugated to therapeutic or diagnostic agents such as radioactive isotopes or anticancer agents.

In one embodiment, the method of the present invention provides a method for treating cancer and thus the isolated antibodies or antigen-binding molecules are conjugated to one or more anticancer agents and are then re-administered to the subject to target cells bearing the selected tumor-associated carbohydrate- or glycopeptide-based antigen. For example, the method involves isolating antibodies or antigen-binding molecules utilizing the inventive affinity matrices, and conjugating one or more therapeutic agents to the isolated antibodies or antigen-binding molecules, and re-administering the conjugated antibodies or antigen-binding molecules to the subject in need thereof.

As described in detail herein, the antibodies or antigen-binding molecules are isolated using an affinity matrix of the present invention having the carbohydrate-or glycopeptide-based antigen (present in either monomeric or clustered form or a mixture thereof) bound to the matrix. The isolated antibodies or antigen-binding molecules are naturally occurring or produced in respect to a tumor associated carbohydrate-or glycopeptide-based antigen vaccine. The vaccine includes, but is not limited to the carbohydrate-based antigens (monomeric and/or clustered) globo-H, Le^{y}, GM2, GD2, GD3, fucosyl GM1, S-Tn, Tn, TF, KH-1, N3, glycosylated segments of muc 1 and muc 2, or combinations thereof. After isolation, therapeutic agents are conjugated to the isolated antibodies or antigen-binding molecules. Therapeutic agents are any suitable substances including, but not limited to, radioactive isotopes or anti-cancer drugs. These conjugated antibodies are then re-administered to the subject. The conjugated antibodies or antigen-binding molecules then seek out and bind to the tumor cell-associated carbohydrate- or glycopeptide-based antigen and deliver the therapeutic substances. Re-treating patients with their own purified antibodies as radio-labeled or drug substituted conjugates simplifies the technical difficulties involved in (humanizing) mouse monoclonal antibodies and the regulatory limitations of using human antibodies derived from other sources, as well as provide new insights into cancer and anti-cancer vaccine therapy. Suitable radioisotopes to be used include, but are not limited to, ¹³¹I, ¹²⁵I, ¹¹¹In or ^{99m}Tc. Goldenberg, D. M., Am. J. Med. 94:297-312, 1993; Jurcic, J. G. and Scheinberg, D. A. Curr. Opin. Immunol. 6;715-721, 1994. Any number of therapeutic drugs known in the art, preferably those approved by the FDA, such a doxorubicin, can be used to construct drug-antibody conjugates. Trail, P. A., Willner, D, Bionchi, A. B., Henderson, A. J., Trailsmith, M. D., Girit, E., Lach, L. S., Hellstrom, I., and Hellstrom, K. E., Clin. Cancer Res. 5:3632-3638, 1999. Other approved chemotherapeutic drugs, include, but are not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In other embodiments, the method of the present invention provides a method for imaging cancer metastases in a subject having cancer, wherein the tumor cells express carbohydrate-or glycopeptide-based tumor antigens. According to the method of the invention, isolated antibodies or antigen-binding molecules are conjugated to one or more diagnostic agents, such as radioactive isotopes, and then are re-administered to the subject. Diagnostically, these isolated, functional antibodies allow detection of early forms of cancer, assessment of patient prognosis to determine treatment, imaging metastases with radiolabeled antibodies, or monitoring the progress of a patient being treated.

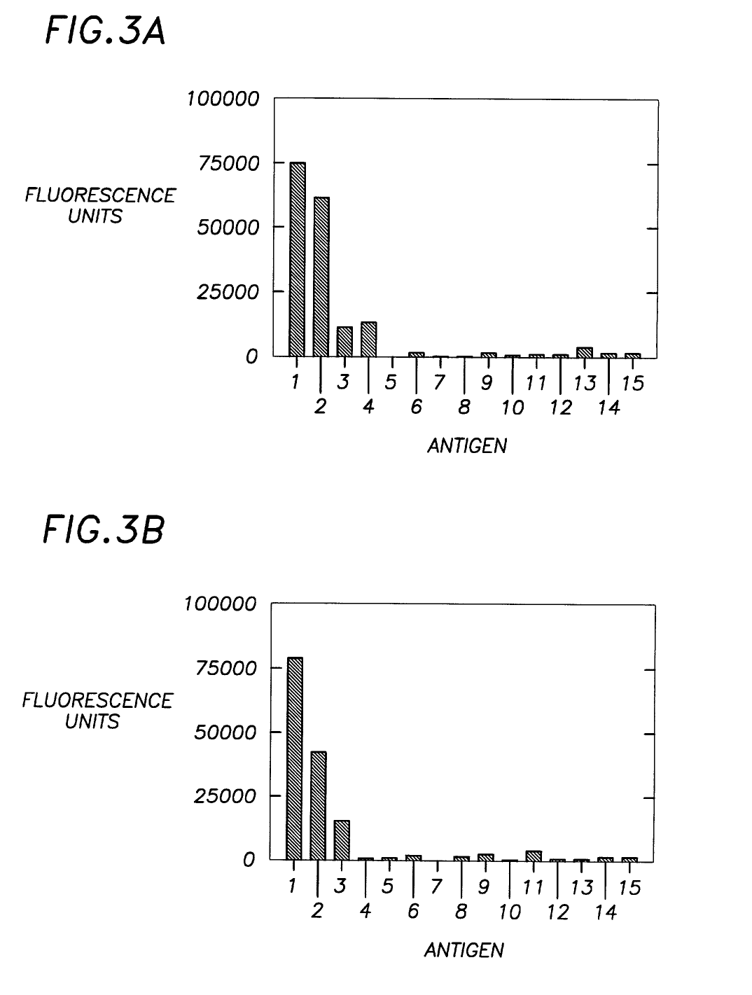

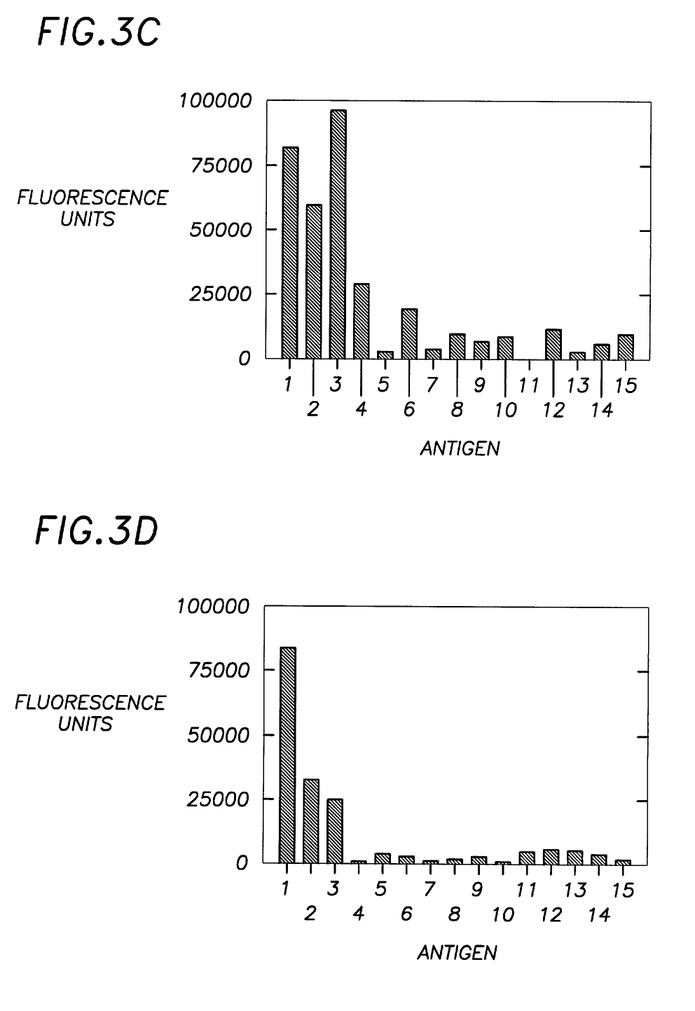

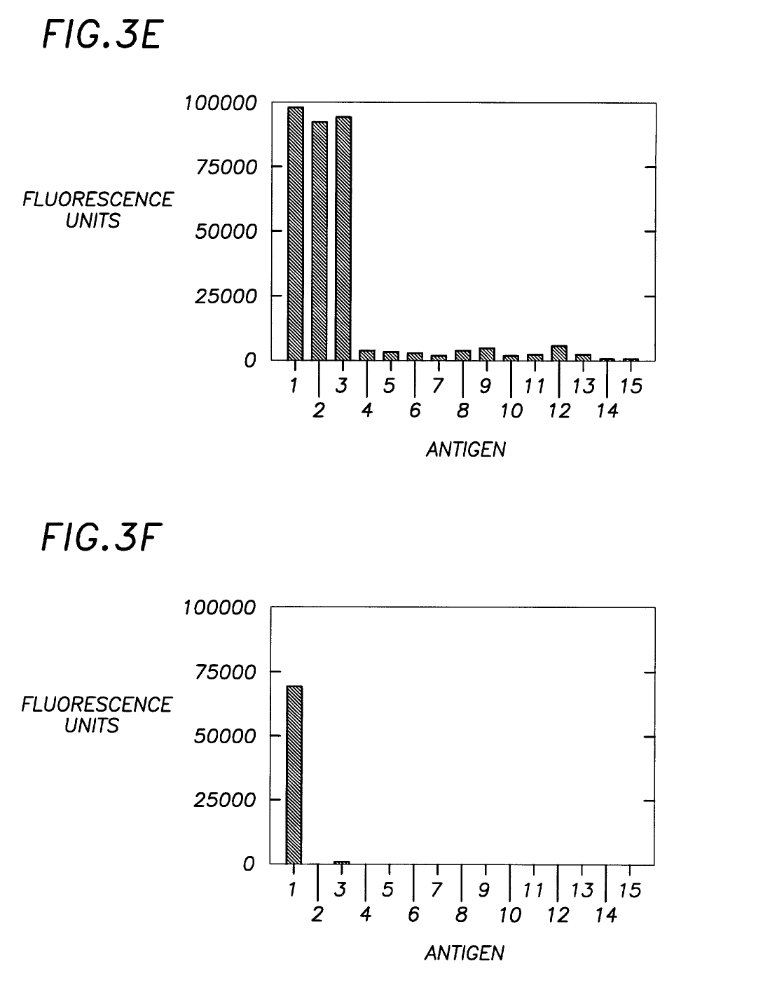

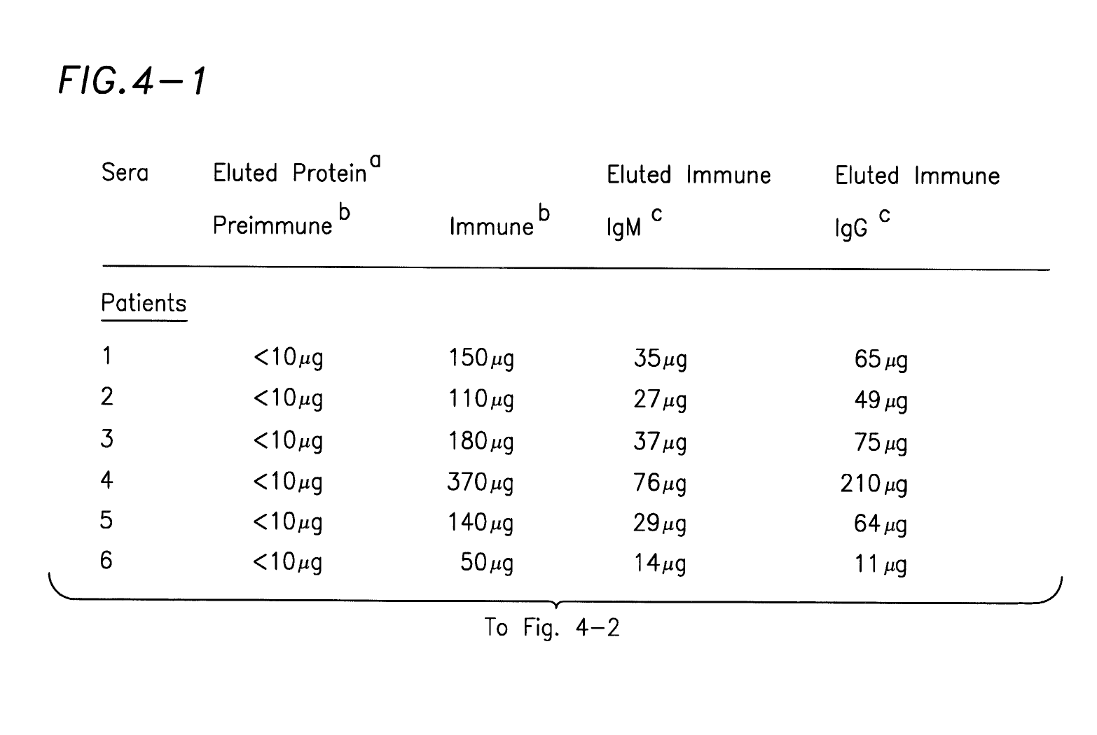

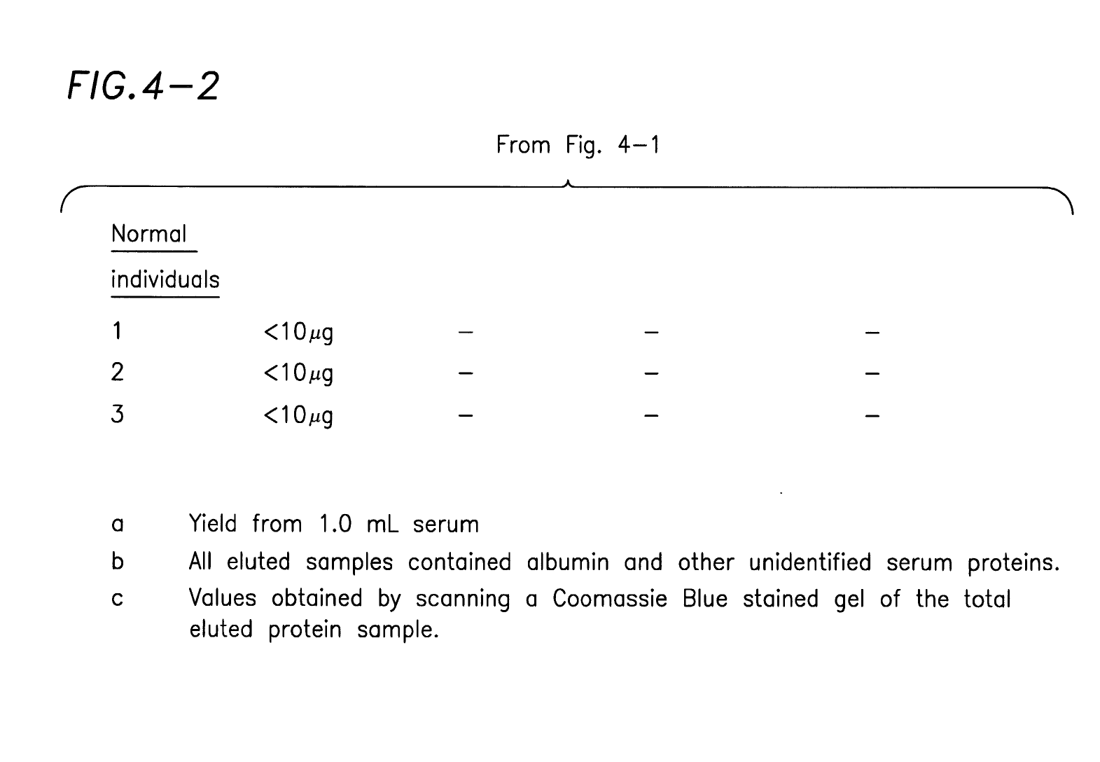

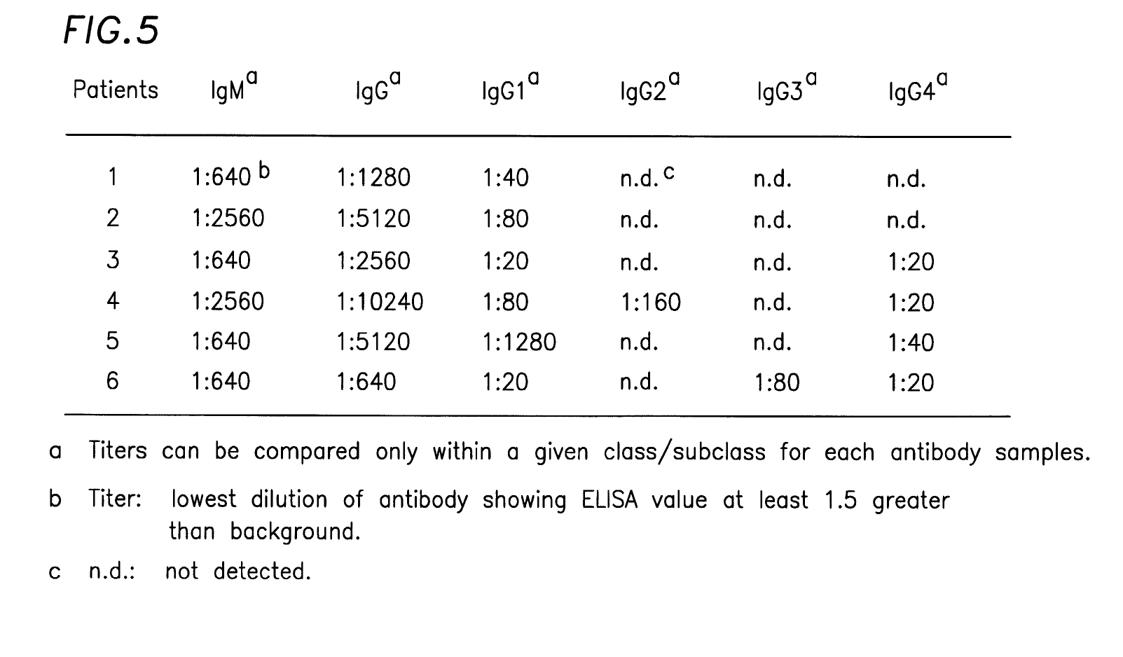

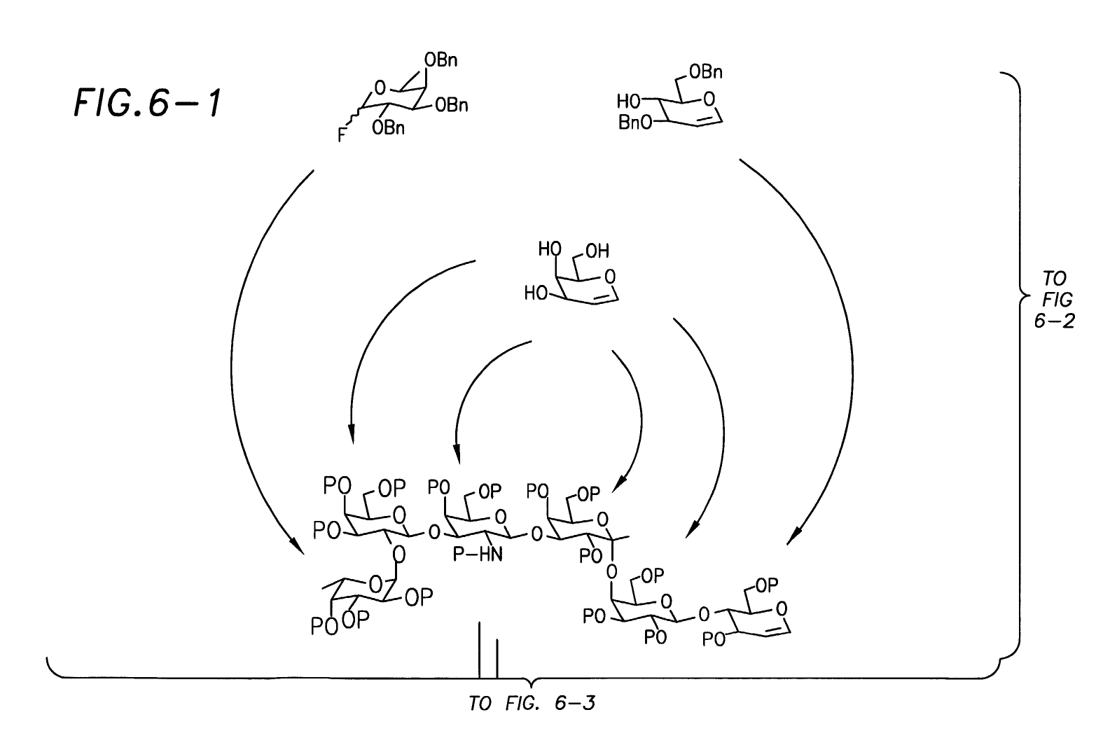

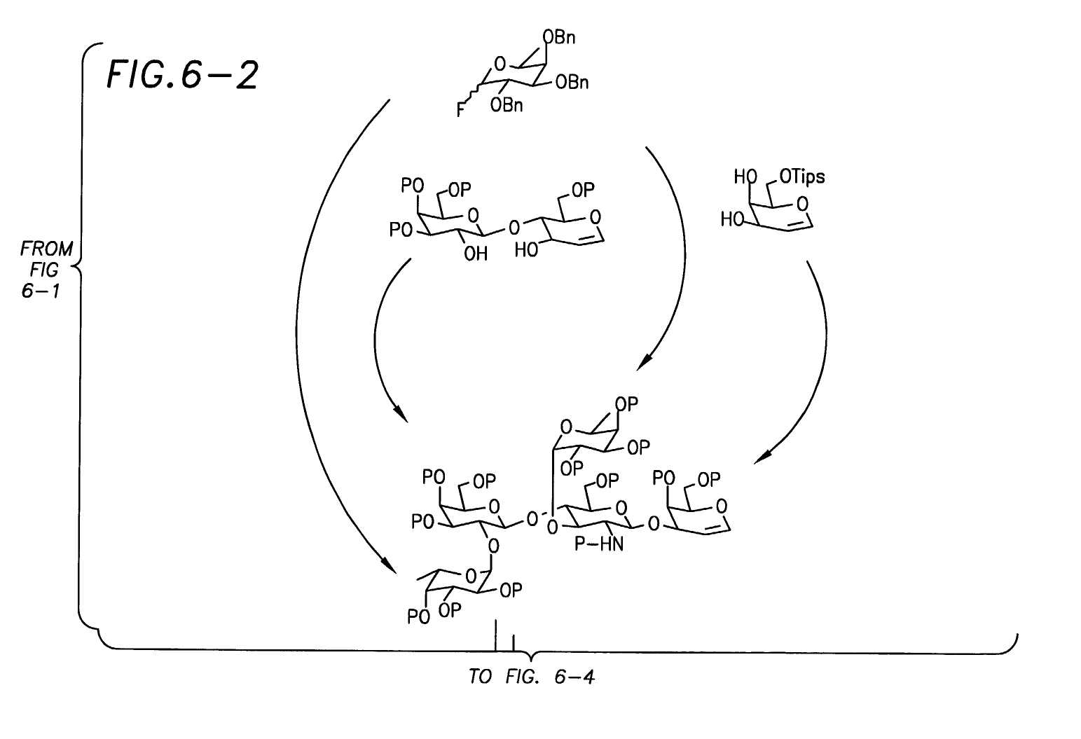

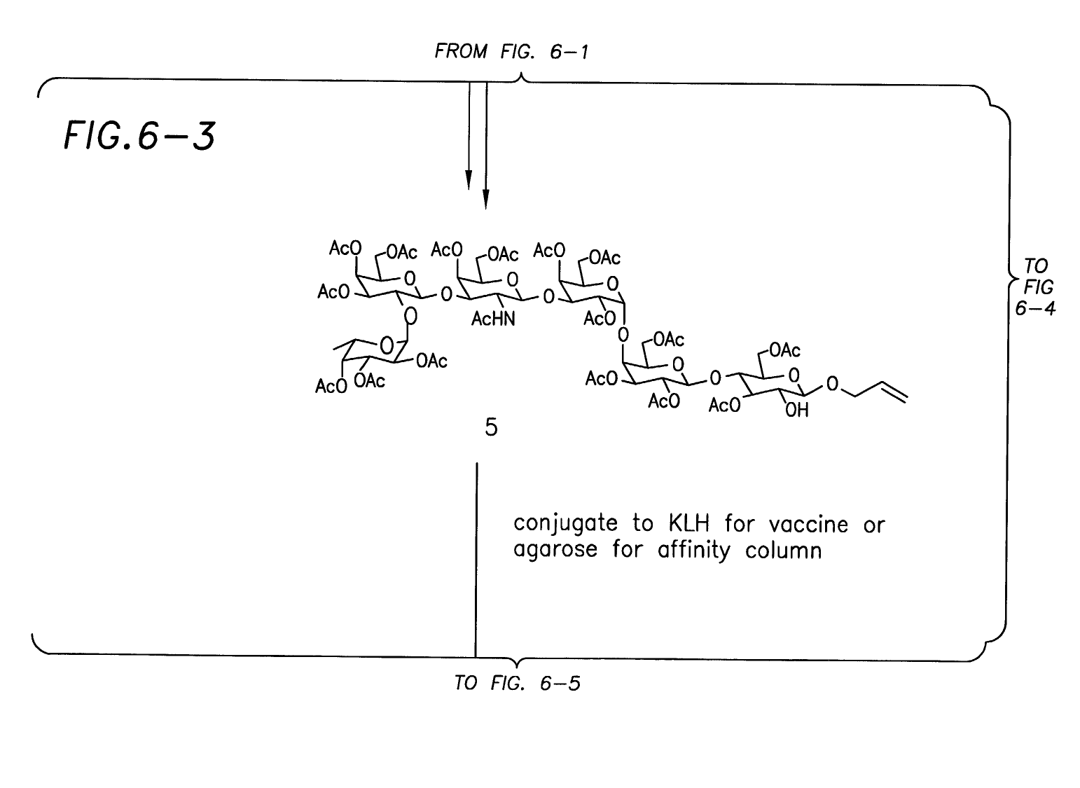

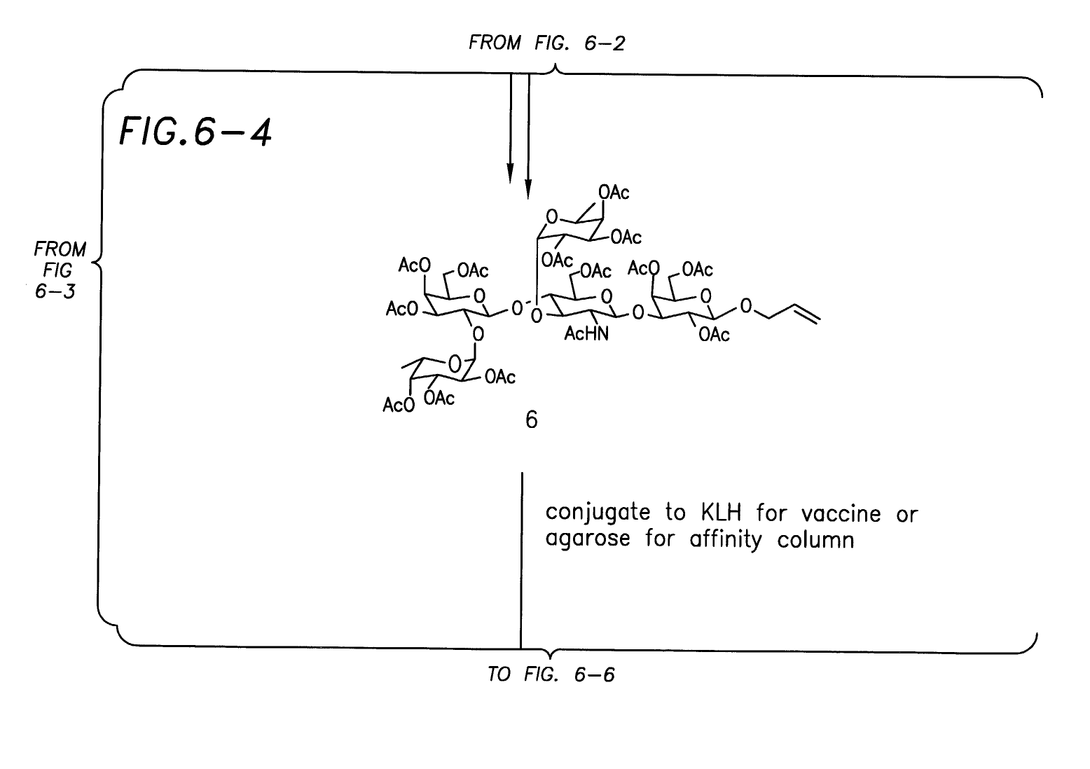

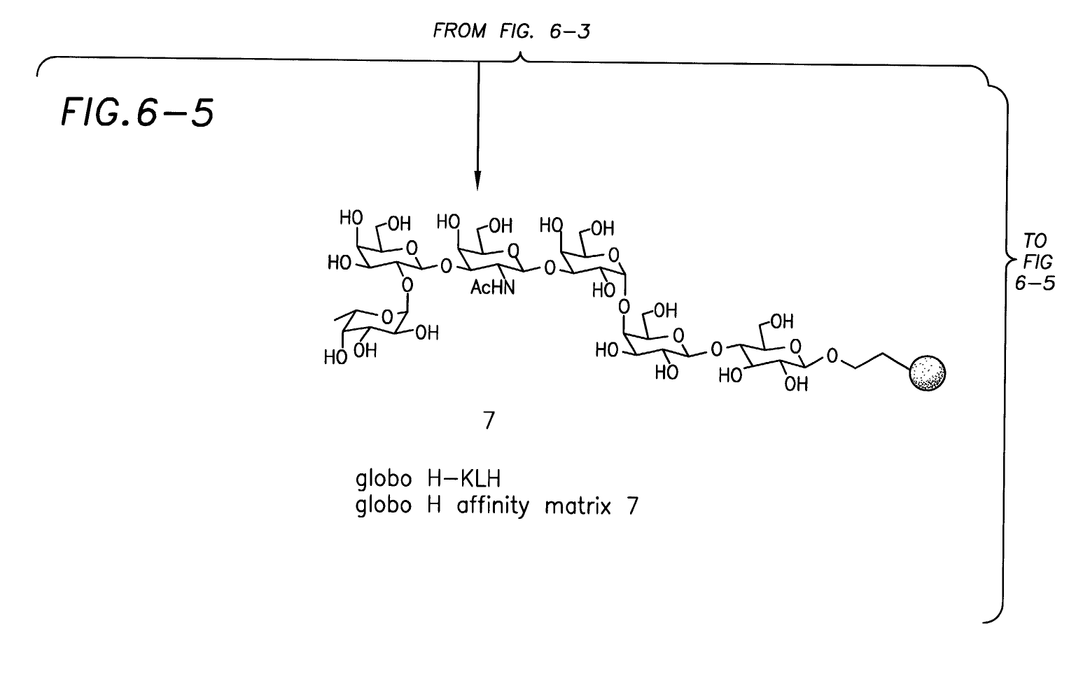

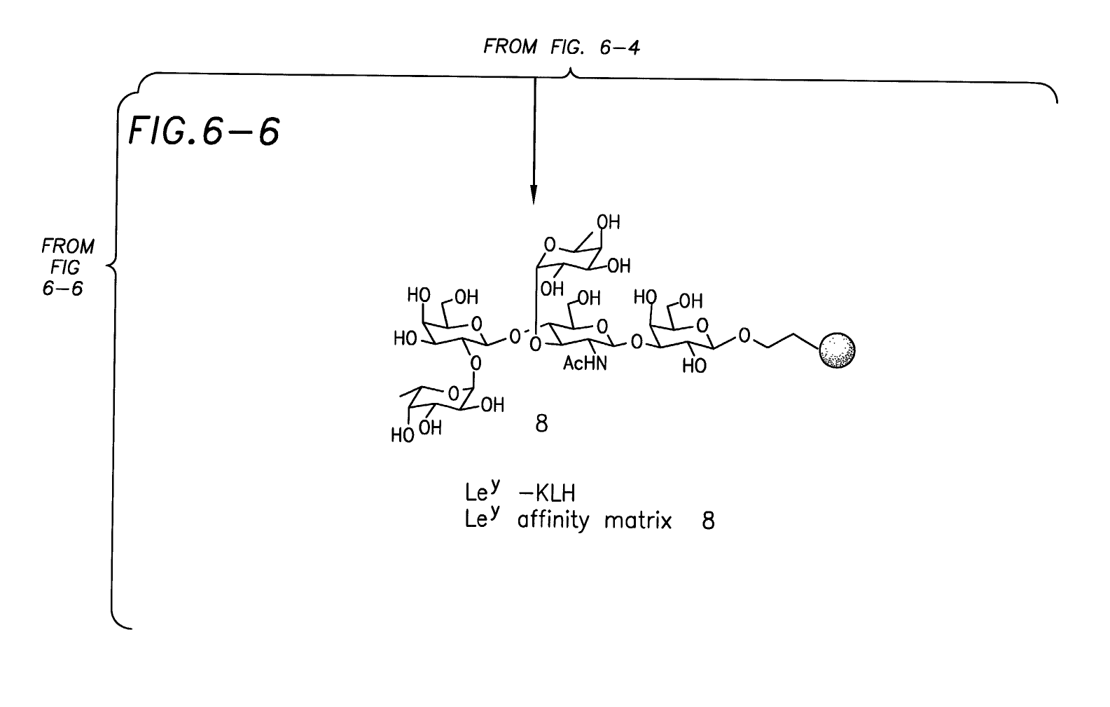

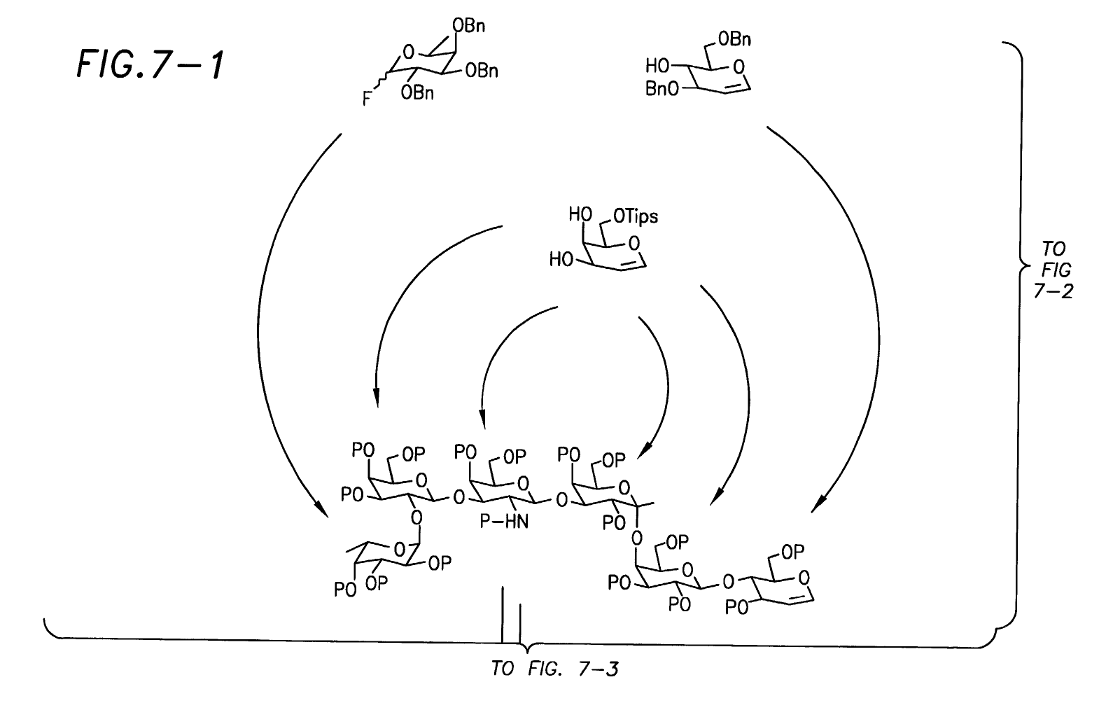

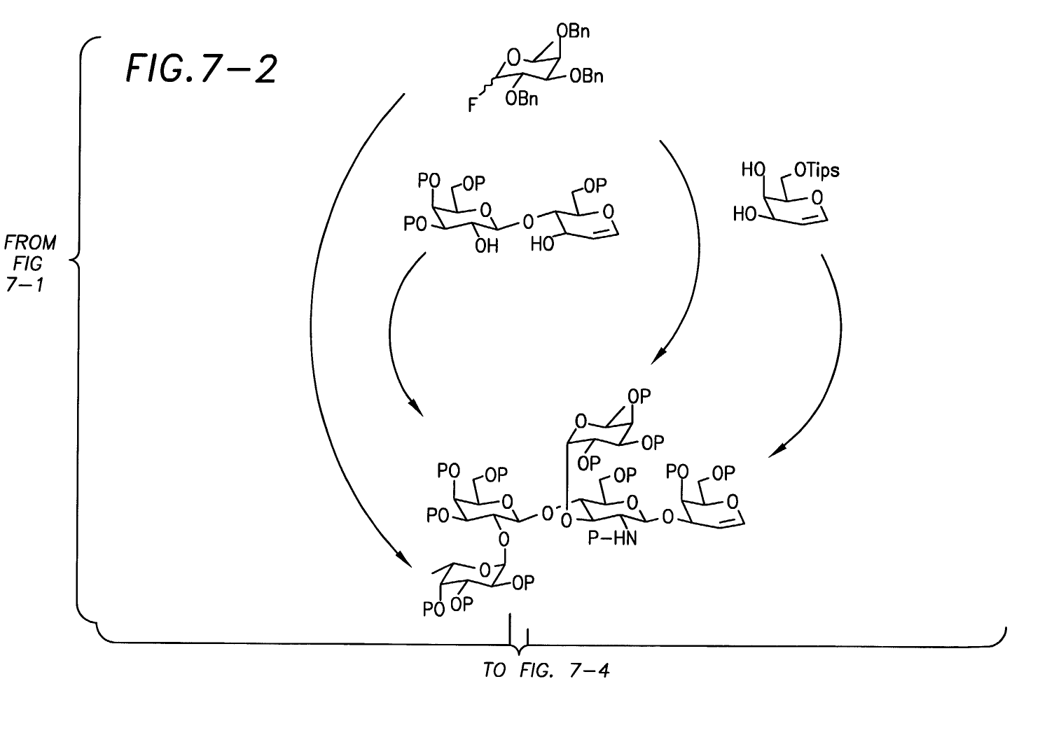

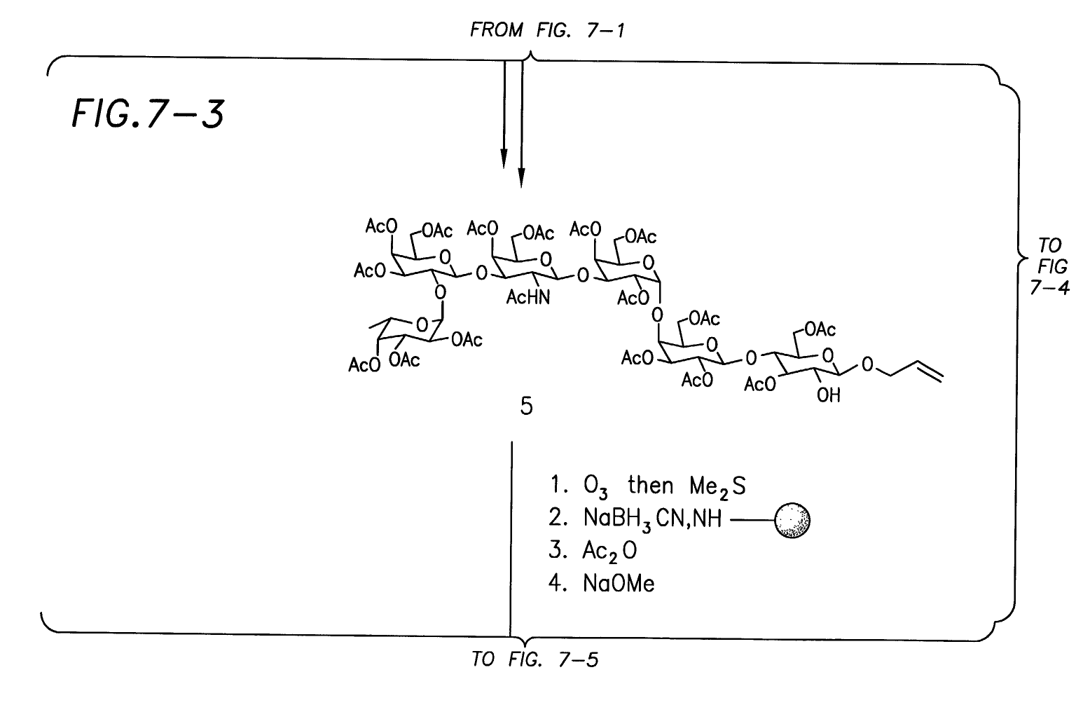

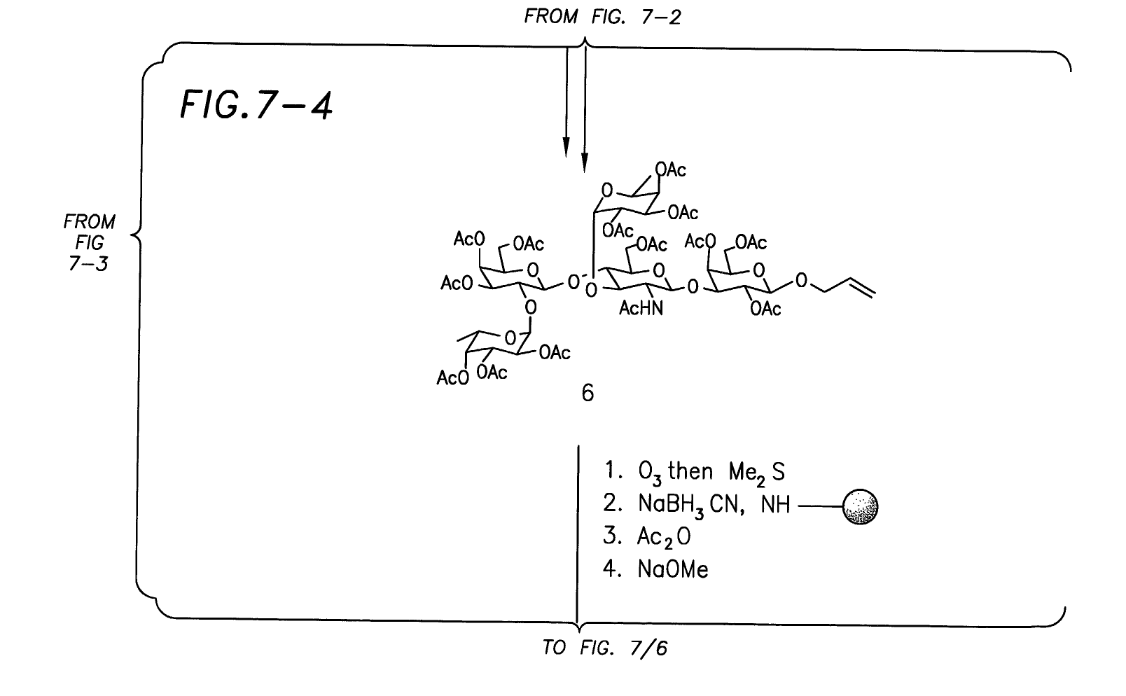

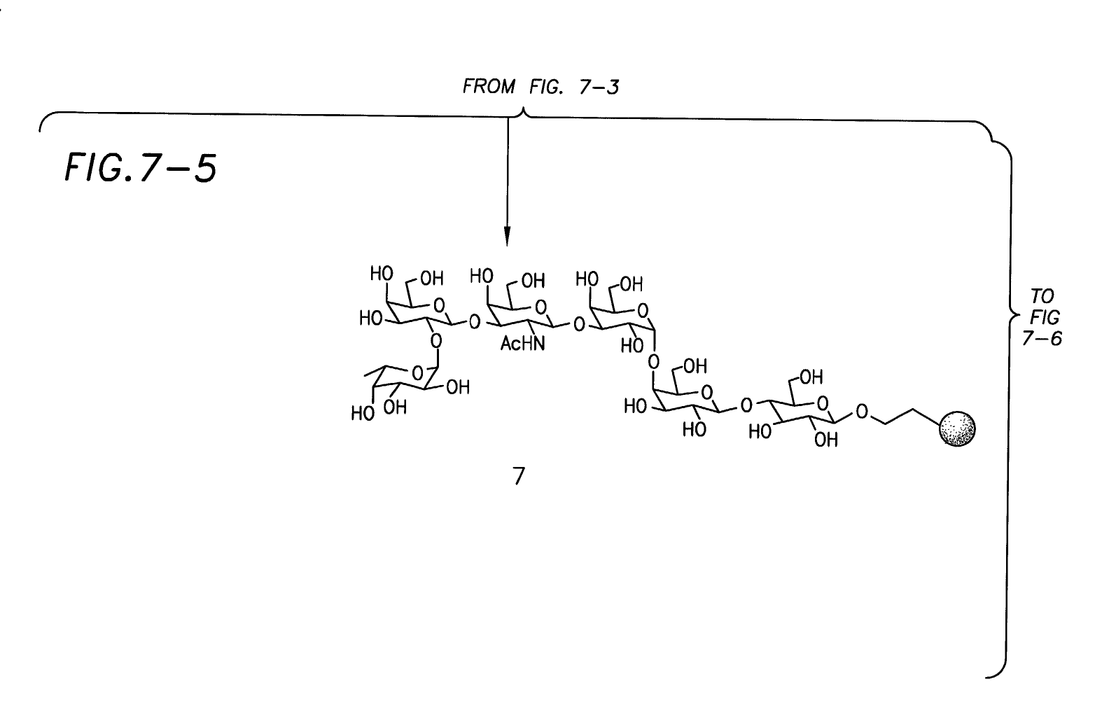

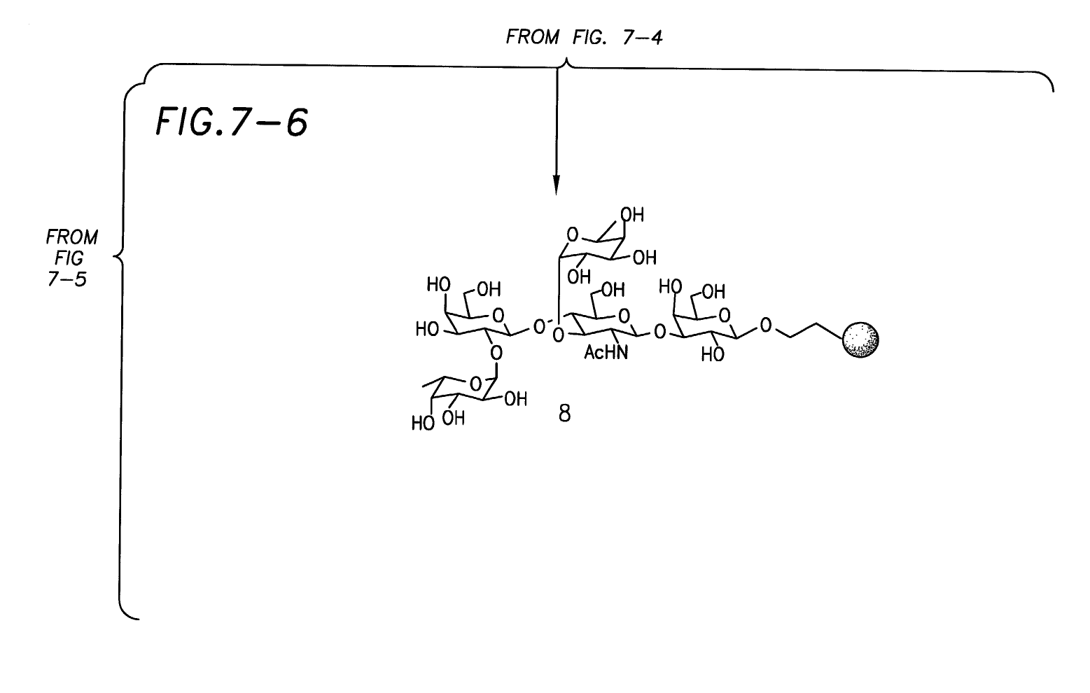

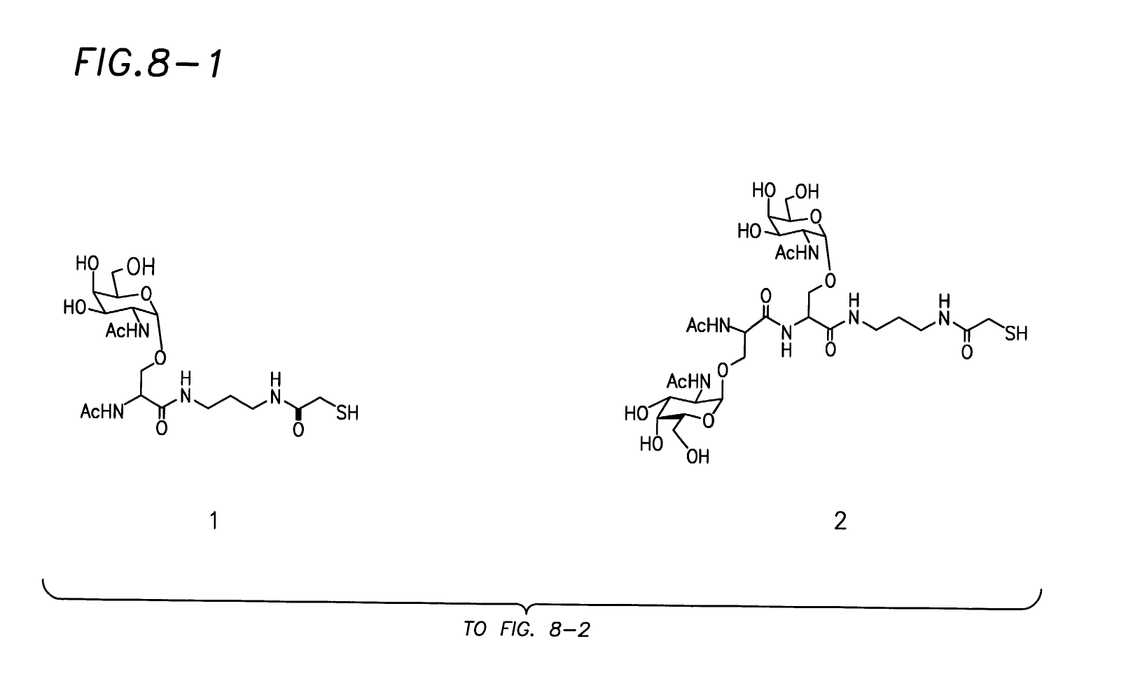

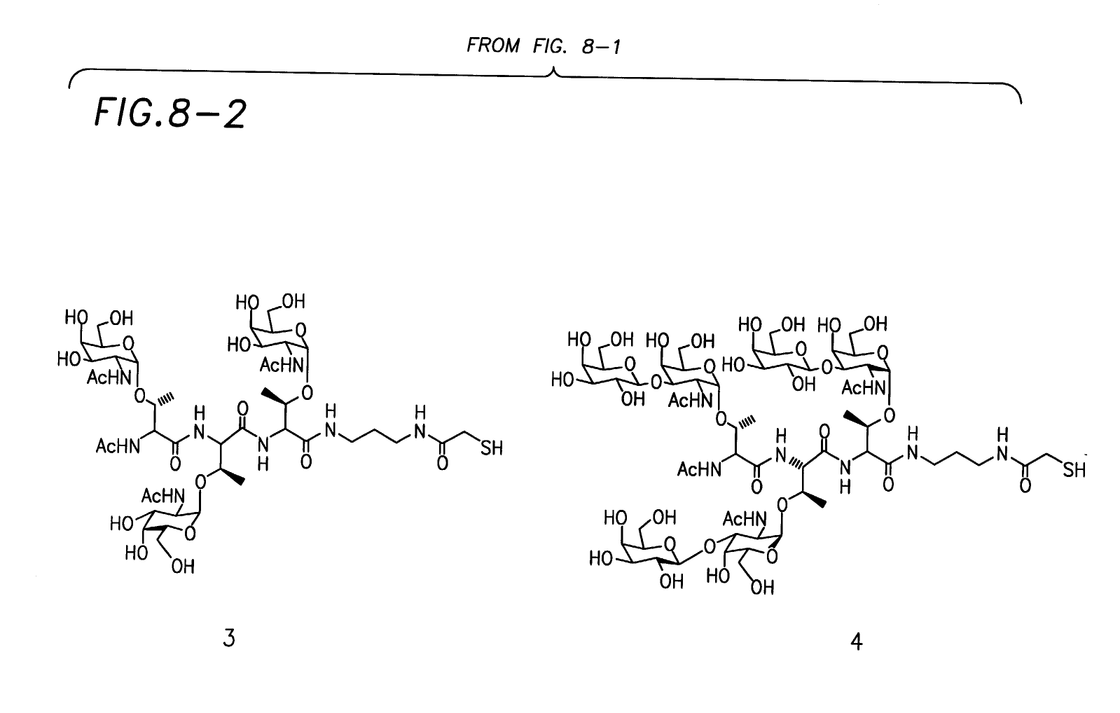

The present invention also provides a method of detecting and diagnosing a cancer in a subject where the cancer cells have tumor-associated carbohydrate-or glycopeptide-based antigens. This method involves providing blood fluids from a subject to an affinity matrix having a tumor-associated carbohydrate-or glycopeptide-based antigen (monomeric and/or clustered) bound to the matrix and washing the matrix to remove unbound substrates. The matrix is then treated with a suitable solution, such as a mild glycine hydrochloride solution, to elute antibodies or antigen-binding molecules from the matrix. The presence of antibodies or antigen binding molecules in the subject's blood fluids indicates the presence of a cancer having tumor-associated carbohydrate-or glycopeptide-based antigens. For example, pre-immune sera and sera from individuals with no history of cancer is applied to the matrix of the present invention having a globo-H oligosaccharide bound to the matrix. The sera from the individuals prior to immunization with the globo-H vaccine and the sera from individuals with no history of cancer do not contain antibodies or antigen-binding molecules that bind to the globo-H affinity matrix. Post vaccination sera from these individuals show antibodies to the globo-H antigen which bind to the affinity column. (compare FIG. 3, panels A, B, and C). This method of detection or diagnosis is applicable to any cancer having a tumor-associated carbohydrate-or glycopeptide-based antigen, such as, but not limited to, cancers expressing monomeric or clustered globo-H, Le^{y}, GM2, GD2, GD3, fucosyl GM1, S-Tn, Tn, TF, or glycosylated segments of muc 1 and muc 2 antigens.

The present invention also provides for monitoring the treatment of cancer. The blood fluids of a patient undergoing treatment for a cancer having tumor-associated carbohydrate-or glycopeptide-based antigens are applied to an affinity matrix having the respective tumor-associated carbohydrate-or glycopeptide-based antigen (monomeric and/or clustered) bound to the matrix. Effectiveness of the treatment is indicated by monitoring the presence and/or quantity of antibodies are antigen-binding molecules to the tumor-associated carbohydrate- or glycopeptide-based antigen in the subject's blood fluids. Any therapeutic treatment for a cancer having a tumor associated carbohydrate- or glycopeptide-based antigen can be monitored. The cancer being treated can be any cancer having a tumor-associated carbohydrate- or glycopeptide-based antigen and is preferably a cancer having a tumor-associated carbohydrate- or glycopeptide-based antigen and is preferably a cancer having a globo-H, Le^{y}, GM2, GD2, GD3, fucosyl GM1, S-Tn, Tn, TF, or glycosylated segments of muc 1 and muc 2 antigen. These cancers include prostrate, breast, ovarian, pancreatic, melanoma, neurobastoma, and small cell lung cancer. It will be appreciated that the treatment can be monitored at specific time intervals (e.g., once a month, once a week) for a selected duration (over the course of one year or over the course of two years, for example).

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and the equivalents thereof.

### Exemplification

The examples listed below are illustrative and are not intended to limit the scope of the invention.

### EXAMPLE 1

### Preparation of Globo-H Affinity Column

Allyl glycoside globo-H hexasaccharide is prepared as described in Park, T. K., Kim, I. J., Hu, S, Bildeau, M. T., Randolph, J. T., Kwon, O & Danishefsky, S. J., J. Amer. Chem. Soc., 118, 11488-11500 (1996). Ozone is passed through a solution of globo-H hexasaccharide (52 mg, 0.030 mmol) in MeOH (10 ml) at −78° C. The reaction is monitored by TLC using Ch₂CI₂/MeOH (10:1) as eluent. The typical reaction time is 5 minutes. To remove excess ozone, the reaction is purged at −78° C. with a stream of N₂ upon disappearance of the starting material followed by the addition of dimethyl sulfide (10 ml). The resulting solution is allowed to warm to room temperature and stirred for a total of three hours. The crude material is concentrated with a stream of N₂ and is used immediately for conjugation to agarose.

The crude ozonolysis product was taken up in MeOH (20 ml) and transferred to a flask charged with amino agarose (Bio-Rad, 10 ml gel, 16.29 μmol/ml), which has been pre-equilibrated with MeOH (2×10 ml). The resultant slurry is treated with NaBH₃CN (1 M, 120 μl, 4 eq) and mixed by vigorous agitation overnight at room temperature. The solvent is removed by filtration and the polymer is washed with MeOH (2×10 ml). The derivatized agarose is treated with acetic anhydride (50 ml) in MeOH (10 ml) for 30 minutes to ensure all amine functions are Negative Ninhydrin test on <1 mg of material is used to verify lack of free amine functionality. To deprotect the agrose-bound carbohydrate antigen, the functionalized matrix is washed with MeOH (2×10 ml) and treated with NaOMe (25% in MeOH, 300 μl) in MeOH (10 ml) for 12 hrs with vigorous agitation at room temperature. The polymer is washed with MeOH (3×10 ml), isopropanol (3×10 ml) and finally with 0.05% aqueous NaN₃ (2×10 ml) providing 10 ml of affinity column material globo-H bound agarose. The loading is determined to be 5 μg fucose/50 μl gel as determined by fucose analysis of the functionalized gel. Lloyd, K. O. & Savage, A., Glycoconjugate J. 8, 493-498 (1991).

### EXAMPLE 2

### Preparation of Le^{y} Agarose Affinity Column

Protected Le^{y}-allyl glycoside is prepared as described in Behar, V. & Danishefsky, S. J., Angew Chem. Int. Ed. 33, 1468-1470. Le^{y}-agarose is prepared following the same procedure as above with 20 mg of Le^{y} pentasaccaharide and 4 ml of amino agarose gel. The loading is determined to be 2.7 μg fucose/50 μl gel as determined by fucose analysis of the functionalized gel. Lloyd, K. O. & Savage, A., Glycoconjugate J. 8, 493-498 (1991).

### EXAMPLE 3

### Glycopeptide Affinity Columns: General Preparative Techniques

A) General Procedure for the Preparation of MBS-Activated Amino Agarose.

Amino functionalized agarose (10 mL,10-16 μmol/mL) is washed with dimethyl formamide (3×10 mL) and to this m-malemidobenzoyl N-hydroxysuccinimide (MBS) (50-200 mg) in approximately 1 mL of N,N-dimethyl formamide is added in one portion. The mixture is allowed to react, with gentle agitation, for 2-12 hours at which time the derivatized agarose is washed with N,N-dimethyl formamide (3×10 mL), methanol (3×10 mL) and phosphate buffered saline (pH 7) (3×10 mL). The MBS-activated agarose is used immediately for the preparation of a glycopeptide affinity column.

B) General Procedure for the Preparation of Glycopeptide Affinity Columns

Glycopeptide Affinity Columns bearing any one of (1-4) were prepared according to the general procedure as follows: Any one of fully deprotected glycopeptide conjugates (1-4) (See FIG. 8 (1-4), 5-25 mg), or any other suitable glycopeptide prepared as described herein, are taken up in aqueous solution (1 mL) containing dithiothreitol (7.7 mg) and agitated at room temperature for 24 hrs. The mixture is then added directly to a size exclusion column (Sephadex G10, Sigma) and eluted with PBS at pH 7.0 and collected in fractions of approximately 1 mL volume. Using approximately 10 μL from each fraction so collected, Ellman's reagent is used to determine the presence of thiol containing compounds. In this way the elution of the pure, fully reduced glycopeptide is efficiently separated from dithiothreitol reducing agent and by-products. Verification of the carbohydrate in thiol-positive fractions (usually the first set) is conveniently confirmed by TLC staining with orcinal followed by charring. Fractions containing glycopeptide are pooled and added to MBS-activated amino agarose (10 mL) and agitated for 24 hrs at RT. After washing the column material with water (3×10 mL), the glycopeptide bearing affinity column is obtained and ready for use. The matrix material is stored at 4° C. in 0.05% NaN₃(aq).

### EXAMPLE 4

### Glycopeptide Affinity Columns Containing Tn Antigen: Preparative Techniques

A) Procedure for the Preparation of MBS-Activated Amino Agarose Prior to Coupling with 2 (See, FIG. 8).

Amino functionalized agarose (10 mL, 13 μmol/mL) was washed with dimethyl formamide (3×10 mL) and to this m-malemidobenzoyl N-hydroxysuccinimide (MBS) (122 mg) in 0.7 mL of N,N-dimethyl formamide was added in one portion. The mixture was allowed to react, with gentle agitation, for 2 hours at which time the derivatized agarose was washed with N,N-dimethyl formamide (3×10 mL), methanol (3×10 mL) and phosphate buffered saline (pH 7) (3×10 mL). The MBS-activated agarose was used immediately for the preparation of an affinity column bearing glycopeptide 2.

B) Procedure for the Preparation of Affinity Column Derivatized with Glycopeptide 2 (See, FIG. 8).

The fully deprotected glycopeptide conjugate 2 (10 mg) was taken up in aqueous solution (1 mL) containing dithiothreitol (7.7 mg) and agitated at room temperature for 24hrs. The mixture was then added directly to a size exclusion column (Sephadex G10, Sigma) and eluted with PBS, pH 7.0, and collected in fractions of approximately 1 mL volume. Using approximately 10 μL from each fraction so collected, Ellman's reagent was used to determine the presence of thiol containing compounds. In this way the elution of the pure, fully reduced glycopeptide was separated from dithiothreitol reducing agent and by-products. Verification of the carbohydrate in the first set of thiol-positive fractions was confirmed by TLC staining with orcinal followed by charring. Fractions containing glycopeptide were pooled and added to MBS-activated activated amino agarose (prepared as described above) and agitated for 24 hrs at RT. After washing the column material with water (3×10 mL), the glycopeptide bearing affinity column was obtained. The matrix material was stored at 4° C. in 0.05% NaN₃(aq).

### EXAMPLE 5

### Isolation of Antibodies by Affinity Chromatography

A globo-H or Le^{y}-agarose column (3.0 ml), or any other suitable affinity matrix, as described herein, is first equilibrated in PBS (20 ml, 0.15 M NaCl, 0.02M phosphate buffer, pH 7.2). The serum to be analyzed (1.0 ml) is then added to the column and allowed to react for 1 hour by agitating gently at 4° C. Subsequently the column is washed with (i) PBS (10 ml) and (ii) 1M NaCl in PBM (5 ml). The antibodies are eluted from the column with 0.05 M glycine-HCl, pH 2.5 (10 ml), and fractions (1.0-2.0 ml) are collected. The samples eluted with the third buffer are collected directly into 75 μl saturated Na₂HPO4 to give a final pH of 6.5-7.5. The fractions are assayed for the presence of protein by monitoring optical density at 280 nm and for antibody activity by ELISA (see Example 6). Fractions showing antibody activity are pooled and used for further analysis. The columns are used repeatedly after washing in the glycine buffer (30 ml) and re-equilibration in PBS. To remove HSA from the eluted fractions, the samples are reapplied to a globo-H-agarose column and washed with PBS (10 ml), 1% NP40-PBS (10 ml), and PBS (10 ml) before eluting the antibody with glycine-HCl buffer (10 ml) as previously described.

### EXAMPLE 6

### Enzyme-linked Immunosorbent Assay (ELISA)

ELISA is performed by methods known in the art. Kudryashov, V., Ragupathi, G., Kim, I. J., Breimer, M. E., Danishefsy, S. J., Livingston, P. O. & Lloyd, K. O., Cancer Immunol. Immunother, 45,281-286 (1998). Briefly, wells of Terasaki 60 well microtiter plates (Nunc 162118) are coated with globo-H (or other test antigens) by allowing the solvent to evaporate at room temperature. After blocking with 2% bovine serum album (BSA)-PBS, aliquots of diluted antiserum are added and allowed to react at room temperature for 1 hour. Subsequently, the plates are washed three times with 0.5% BSA-PBS. Bound antibody is quantitated with an appropriate alkaline phosphatase-coupled anti-Ig reagent: rabbit anti-human IgG (1:500; Sigma Chemical Co., St. Louis, Mo.) for human sera and rabbit anti-mouse IgG (1:500; Sigma Chemical Co., Mo.) for mouse antibodies. In some experiments p-nitrophenylphosphate (1 mg/ml) is used as the enzyme substrate. In later experiments, reactivity is assayed with fluorescein phosphate (Molecular Probes, Inc., Eugene, Oreg.; 0.05 mM in 0.1M Tris pH 9.9, 50 mM NaCl, 10 mM MgCl₂ and 0.1mM ZnCl₂) and quantitation in a fluorescence plate reader with excitation at 485 nm and emission at 535 nm (Wallach, Model 1420). For determination of IgG subclass, alkaline phosphatase-coupled anti-IgG1, -IgG2, -IgG3 and -IgG4 specific antibodies (Southern Biotechnology, Inc.) are used in the final step.

### EXAMPLE 7

### SDS-Polyacrylamide Gel Electrophoresis (SDS-PAGE)

SDS-PAGE in 7% acrylamide gels is carried out under non-reducing conditions in the absence of 2-mercaptoethanol as described, Yin, B. W. T., Finstad, C. L., Kitamura, K., Federici, M. G., Welshinger, M., Kydryashov, V., Hoskins, W. J., Welt, S. & Lloyd, K. O., Int. J. Cancer 65, 406-412 (1996).

### EXAMPLE 8

### Identification and Quantification of Antibodies Produced in Response to Vaccination

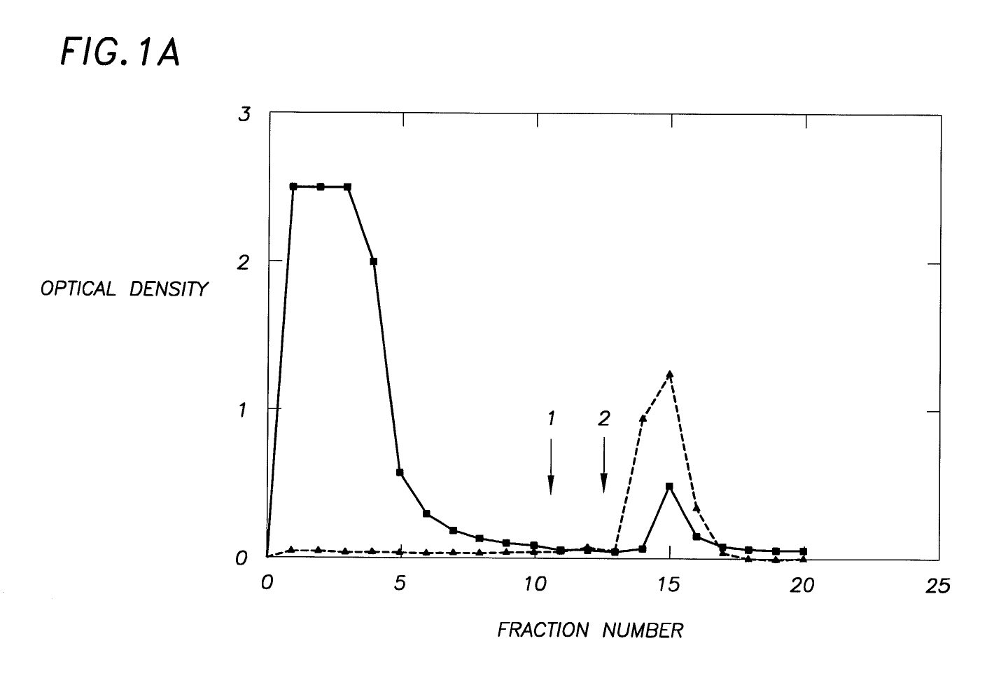

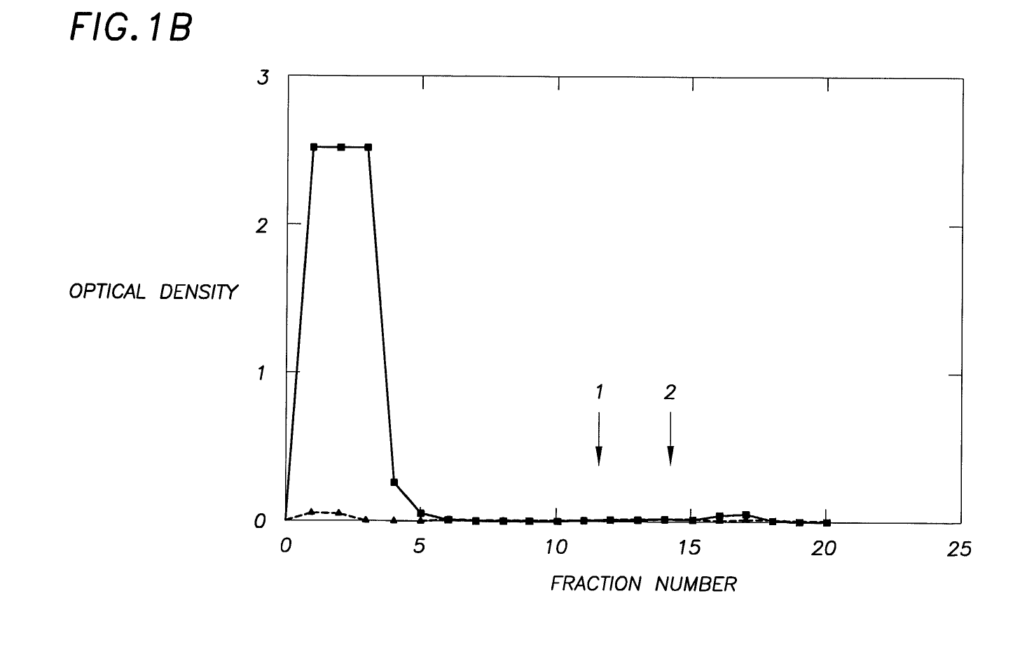

Sera from six patients who had been immunized with a globo-H-KLH vaccine and sera from three normal individuals are fractionated on a globo-H-agarose affinity column as described in Example 4. As assayed by ELISA, all anti-globo-H antibody activity in the sera of immunized patients is retained by the column. Elution with high salt (1M NaCl) does not remove antibody from the column but elusion with a glycine-HCl, pH 2.5 buffer results in the elution of antiglobo-H antibody. (FIG. 1, Panel A). Sera obtained from the same patients before immunization, or from normal individuals, contain undetectable, or only trace quantities, of antibody. (FIG. 1, panel B). The columns of the present invention maintain high specificity for the desired antibody. For example, the specificity of anti-globo-H binding is demonstrated with the Le^{y} column, in that no ‘cross talk’ occurred. Thus, antibodies from patients vaccinated with globo-H-KLH bind to the globo-H column. However, the anti-globo-H antibodies do not bind to the Le^{y} affinity column but Le^{y}-KLH vaccinated patient sera contain antibodies that bind to the Le^{y}-agarose affinity column. Sabbatini, P. J., et al. (submitted).

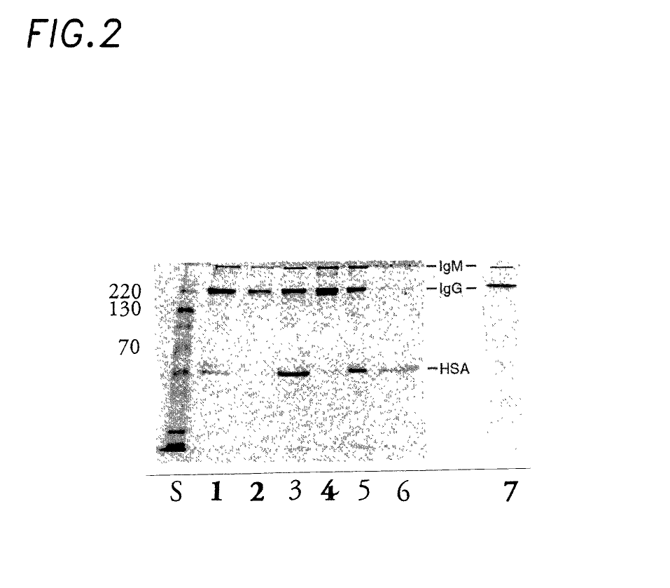

Pooled fractions from the six anti-globo-H sera are analyzed for protein content using the Lowry assay (FIG. 4) and by SDS-PAGE electrophoresis for purity (FIG. 2). The total protein content of the eluted fractions ranges from 50-370 μg/mL serum. However, SDS-PAGE analysis shows that in addition to IgG and IgM immunoglobulins some of these samples contain substantial amounts of a component migrating with human serum albumin (HSA). The identity of this component is confirmed to be HSA by Western blotting with anti-HSA antibody. The proportion of IgM and IgG immunoglobulins in the sera is estimated by scanning the Coomassie Blue-stained gel in a Biorad GS700 scanner and Quantity One data analysis system. (FIG. 4).

Successful purification of the antibody from the HSA is achieved by applying the sample to a globo-H-agarose column and washing the column with PBS and then 1%NP40-PBS, (which removed the albumin) before eluting the antibody in glycine pH 2.5 buffer. SDS-PAGE analysis of a typical purified sample is shown in FIG. 2, lane 7.

The presence of both IgM and IgG antibodies in the purified antibody samples, as detected by SDS-PAGE (FIG. 2), is confirmed by ELISA using specific antibodies. (FIG. 5). Subclass analysis with anti-IgG subclass antibodies reveals that IgG1 antibodies are detected in all six samples and that IgG2, IgG3, and IgG4 antibodies are detected in one or more of the samples. (FIG. 5).

The specificity of the eluted antibodies from 5 of the 6 patients is analyzed by direct ELISA on a panel of 15 glycoconjugates. (FIG. 3). As expected, antibody fractions from the five patients react with globo-H (Lane 1). This confirms that the antibodies retain their functionality. Significant reactivity with other targets, which differed substantially between the patients, is also evident. (Compare lanes 4-15 with patients A-G). The antibodies from all five patients cross-react to some extent with galactosyl-globoside (SSEA-3) and globoside (Lanes 2 & 3 respectively). Cross reaction of antibodies with related antigen structures is commonly observed. For anti-carbohydrate antibodies, these reactivities are normally focused on shared non-reducing terminal structures. Furthermore, in two patients the reactivity with these two structures is as high, or almost as high, as with globo-H itself. (FIG. 3, panels C and E). Humans immunized with the globo-H-KLH conjugate product IgG antibodies that appear to mainly recognize an epitope area encompassing five non-reducing terminal carbohydrate units when assessed using sera. Ragupathi, G., Slovin, S. F., Adluri, S., Sames, D., Kim, I. J., Kim, H. M., Spassova, M., Bornmann, W. G., Lloyd, K. O., Scher, H. I., Livingston, P. O. & Danishefsky, S. J., Angew. Chem. Int. Ed. 38,563-566 (1999). However, purified polyclonal antibodies from the same patients clearly include internal carbohydrate sequence binding as well. While, Le^{y}-ceramide shows weak reactivity with the sera from three patients (lane 7), the other antigens tested are essentially unreactive. As a positive control, an anti-globo-H mouse monoclonal antibody (VK-9) reacts exclusively with globo-H. (FIG. 3, panel F).

Assessment of antibody response to vaccination with carbohydrates, or other antigens, commonly relies on titers given as unitless quantities. Typically, the titer values are assessed by comparison to background noise or relative to another entity in situ. A number of studies with bacterial polysaccharide vaccines determined the level of the antibody response in weight units (e.g. μg/ml serum) but no published anti-cancer vaccine studies provide such data for comparison. Using the affinity matrix of the present invention, the antibodies from patients immunized with a globo-H-KLH conjugate vaccine are isolated and quantified. The patients respond with the production of 25-280 μg antibody/ml serum. (FIG. 4). As no comparable data are available on antibodies resulting from the vaccination of other cancer patients, these levels are compared with those quoted in a number of studies with bacterial polysaccharide vaccines in which antibody levels have been quantified. In a study on antibodies elicited in adults with a pneumococcal conjugate vaccine, Soininen et al. reported IgG levels of only 0.58-1.33 μg/ml serum (IgM levels were not reported); although Antitila et al. reported the induction of 7.8-57.8 μg/ml IgG in 15 month old children. Soininen, A., Seppala, I., Nieminen, T., Eskola, J. & Kayhty, H., Vaccine 17, 1889-1897 (1999); Antitila, M., Eskola, J., Ahman, H. & Kayhty, H., Vaccine 17, 1970-1977 (1999). Kabat and Berg reported anti-dextran antibodies, mainly in the range of 1.9-97.5 μg/ml serum in adults immunized with dextran polysaccharide (with two individuals with levels greater than 250 μg/ml). The data of Kabat and Berg was presented in μg nitrogen precipitated by dextran from serum. By assuming that dextran contains no nitrogen and that the nitrogen content of Ig is 16% the data was recalculated to determine the range of antibodies in serum. Kabat, E. A. & Berg, D., J. Immunol. 70, 514-532 (1953). Remarkably, these comparisons show that the immunization of cancer patients with a tumor-associated carbohydrate-based conjugate vaccine results in the production of antibody levels similar to, and often exceeding, those formed by immunization with bacterial polysaccharide vaccines. The use of affinity purified antibodies also reveals that substantial quantities of IgG, as well as IgM antibodies, are produced in response to the vaccine. Thus, even though globo-H is apparently expressed to a small extent on normal tissues, it is possible to break tolerance using the conjugate vaccine together with adjuvant and to generate a potent immune response focused against it.

### EXAMPLE 9

### Characterization of the Isolated Antibodies

Substantial levels of IgG and IgM antibodies elicited in response to the globo-H vaccine (or other tumor-associated carbohydrate- or glycopeptide based antigens, as described herein) are isolated using the affinity column of the present invention. The levels of IgG antibodies detected in the purified fractions are not only higher than that determined in whole sera by ELISA, but in fact, a reversal of the relative quantities is observed (approximately 2:1 in sera and 1:2 in purified antibodies, compare FIG. 4 with the following reference). Ragupathi, G., Slovin, S. F., Adler, S., Sames, D., Kim, I. J., Kim, H. M., Spassova, M., Bornmann, W. G., Lloyd, K. O., Scher, H. I., Livingston, P. O. & Danishefsky, S. J., Angew. Chem. Int. Ed. 38, 563-566 (1999); Solvin, S. F. Ragupathi, G., Adler, S., Ungers, G., Terry, K., Kim, S., Spassova, M., Bornmann, W. G., Fazzari, M., Dantis, L., Olkiewicz, K., Lloyd, K. O., Livingston, P. O., Danishefsky, S. J. & Scher, H. I., Proc. Natl. Acad. Sci USA 96, 5710-5715 (1999). Clearly then a significant class switch to IgG antibodies is induced. This important finding is not evident from examining antibodies in sera and provides support for the concept of using a protein carrier, such as KLH, to increase the immunogenicity of carbohydrate antigens. These results further demonstrate the utility of the derivatized affinity matrix of the present invention in clinical serological analysis.

The IgG subclass distribution of the anti-globo H antibodies is heterogeneous. It has been believed that anti-carbohydrate antibody responses are restricted to the IgG2 subclass, though exceptions to this rule have been noted. Normansell, D. E., Diag. Clin. Immunol. 5, 115-128 (1987); Livingston, P. O., Ritter, G., Srivastava, P., Padavan, M., Calves, M. J., Oettgen, H. F. & Old, L. J., Cancer Res. 49, 7045-7050 (1989). Analysis of the purified antibodies reveals that all patients respond with the production of IgG1 antibodies. Three patients also produce IgG4 antibodies but only one produces detectable levels of IgG2 (FIG. 5). Thus, the column of the present invention enables the clear determination of the IgG subclass of the antibody response.

Examining the affinity matrix purified antibodies demonstrates unusual subclass population in clinical trials when earlier serial analyses are somewhat ambiguous. Ragupathi, G., Slovin, S. F., Adler, S., Sames, D., Kim, I. J., Kim, H. M., Spassova, M., Bornmann, W. G., Lloyd, K. O., Scher, H. I., Livingston, P. O. & Danishefsky, S. J., Angew. Chem. Int. Ed. 38,563-566 (1999). In this regard, it should be noted that IgG1 antibodies are a subclass known to be able to mediate not only CDC but also ADCC. In previous reports, cell surface reactivity of anti-Globo H antibodies was assessed by flow cytometry, and post-vaccination sera showed strong CDC against MCF-7 cells. See Ragupathi et al. above, and Slovin, S. F. above. These cytotoxicity observations were attributed to the action of antibodies of the IgM class. The low serological reactivity of IgG antibodies, assayed by flow cytometry could be a consequence of low affinity of IgG antibodies for otherwise tolerized molecule, in addition to the typically low affinity of proteins for carbohydrates. This property of IgG antibodies is presumably overcome by the pentavalent nature of IgM antibodies. While not wanting to be limited by theory, these factors are undoubtedly relevant, the more extensive characterization of IgG's reported here with the use of the affinity matrix of the present invention suggest another consideration: HSA binding to immunoglobulins may have obscured both the predominance of IgG1 among antibody constituents and their reactivity in in vitro serial analyses by flow cytometry and CDC assays. These data underscore the inconclusive nature of negative data, as protein analysis is difficult to quantify in the complex setting of serum and highlight the importance of the column of the present invention to allow for a more precise serological evaluation.

### EXAMPLE 10

### Tn(c)-KLH immunized Serum Pass through Tn(c)-column.

The sera from 2 patients immunized with Tn(c)-KLH were combined and added to the column derivatized with 3 (See, FIG. 8) and eluted following the procedure described for the Globo H column. Progress of the purification process was monitored by determination of the optical density (O.D.) of each eluted fraction measured at 280nm, with ELISA, wherein the ELISA plates were coated with Tn(c)-HSA (0.2 μg/well), FACS (using cell of the MCF7 cell line). Pooled sera bound column derivatized with 3 (See, FIG. 8), and purification of all antibody was confirmed by ELISA (both IgG and IgM). FACS data also showed that the purified antibodies exhibited positive reactivity against MCF-7 cell line (See, Table 1)

TABLE 1
Tn(c) Colum vs. Pooled Tn(c)-KLH
serum
plate coated with Tn-HSA Cell: MCF-7

| colum | Fraction | | O.D. | ELISA | | FACS (%) |
|---|---|---|---|---|---|---|
| type | # | | (280 nm) | IgG | IgM | IgG/IgM |
| TnS3 | PBS | 1 | 3 | 0 | 0 | 13.92/88.6 |
| (trimer) | | 2 | 3 | 0 | 0 | 21.52/91.11 |
| vs. | | 3 | 3 | 0 | 0 | 5.04/50.88 |
| Tn(c)- | | 4 | 3 | 0 | 0 | 51.38/20.92 |
| KLH | NaCI | 29 | 0.016 | | | |
| serum. | | 30 | 0.01 | | | |
| | | 31 | 0.009 | | | |
| | glycine- | 32 | 0.013 | 0 | 320 | 1.46/44.51 |
| | HCl | 33 | 0.031 | 10 | 1280 | 1.86/78.37 |
| | | 34 | 0.097 | 320 | | 7.80/87.93 |
| | | | | | 1280+ | |
| | | 35 | 0.03 | 1280 | 320 | 4.80/50.13 |
| | | 36 | 0.023 | 160 | 320 | 3.20/24.29 |
| pre-colum | | | | | | |
| sera | | | | | | /99.2 |
| (Positive | | | | 2560+ | 2560+++ | |
| Control) | | | | | | Cell: LSC |
| TnS3 | PBS | 1 | 3 | 0 | 0 | 9.58/26.70 |
| (trimer) | | 2 | 3 | 0 | 0 | 20.03/54.58 |
| vs. pooled | | 3 | 3 | 0 | 0 | 19.15/12.42 |
| Tn(c)- | NaCl | 24 | 0.009 | | | |
| KLH | | 25 | 0.005 | | | |
| serum(1:4) | | 26 | 0.005 | | | |
| | glycine- | 27 | 0.063 | 0 | 40 | 0.77/1.7 |
| | HCl | 28 | 0.072 | 10 | 320 | 0.85/1.26 |
| | | 29 | 0.187 | 160 | 640 | 1.65/5.03 |
| | | 30 | 0.614 | 160 | 160 | 3.41/2.08 |
| | | 34 | 0.684 | 80 | 10 | 2.36/2.01 |
| | | 35 | 0.726 | 40 | 10 | 1.43/1.50 |
| | | 36 | 0.715 | 40 | 10 | 1.70/1.63 |
| pre-colum | | | | 1280 | 1280+++ | 9.64/98.77 |
| sera | | | | | | |

1. An affinity matrix comprising tumor-associated carbohydrate- or glycopeptide-based antigen bound to the matrix.

2. The affinity matrix of claim 1, wherein the matrix comprises a solid support.

3. The affinity matrix of claim 2, wherein the solid support comprises tentagel, agarose, acrylic or polyacrylamide.

4. The affinity matrix of claim 3, wherein the solid support comprises agarose.

5. The affinity matrix of claim 1, wherein the antigen comprises monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 or muc 2, or combinations thereof.

6. An affinity matrix comprising synthetic tumor-associated carbohydrate- or glycopeptide-based antigen bound to the matrix.

7. The affinity matrix of claim 6, wherein the matrix comprises a solid support.

8. The affinity matrix of claim 7, wherein the solid support comprises tentagel, agarose, acrylic or polyacrylamide.

9. The affinity matrix of claim 8, wherein the solid support comprises agarose.

10. The affinity matrix of claim 6, wherein the antigen comprises monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

11. An affinity matrix comprising monomeric or clustered globo-H-oligosaccharide bound to an agarose support.

12. An affinity matrix comprising monomeric or clustered Lewis Y oligosaccharide bound to an agarose support.

13. An affinity matrix comprising monomeric or clustered Tn bound to an agarose support.

14. An affinity matrix comprising monomeric or clustered TF bound to an agarose support.

15. A method for preparing an affinity matrix comprising the steps of:
a) providing monomeric or clustered tumor-associated carbohydrate- or glycopeptide-based antigen, or a combination thereof; and
b) contacting said carbohydrate- or glycopeptide-based antigen with a solid support, whereby the step of contacting effects binding of the antigen to the support.

16. The method of claim 15, wherein the step of providing monomeric or clustered carbohydrate- or glycopeptide-based antigen comprises providing synthetic monomeric or clustered carbohydrate- or glycopeptide-based antigen.

17. The method of claim 16, wherein the step of providing monomeric or clustered carbohydrate- or glycopeptide-based antigen comprises providing synthetic monomeric or clustered carbohydrate- or glycopeptide-based antigen having terminal allyl functionality.

18. The method of claim 17, wherein the step of providing further comprises converting the terminal allyl functionality to a corresponding in situ aldehyde.

19. The method of claim 15, wherein the step of providing monomeric or clustered carbohydrate- or glycopeptide-based antigen comprises providing synthetic monomeric or clustered carbohydrate- or glycopeptide-based antigen having a terminal allyl, amino, thio or acid functionality.

20. The method of claim 15, after the step of contacting, further comprising the steps of:
capping any residual functionality present on the solid support; and
treating the affinity matrix with a suitable reagent to remove protecting groups present in the support-bound carbohydrate- or glycopeptide-based antigen.

21. The method of claim 15, wherein the step of providing monomeric or clustered tumor-associated carbohydrate- or glycopeptide-based antigen comprises providing globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

22. The method of claim 15 wherein the step of providing comprises providing monomeric or clustered synthetic globo-H-oligosaccharide, or a combination thereof.

23. The method of claim 15 wherein the step of providing comprises providing monomeric or clustered synthetic Lewis Y-oligosaccharide, or a combination thereof.

24. The method of claim 15, wherein the step of providing comprises providing monomeric or clustered synthetic Tn, or a combination thereof.

25. The method of claim 15, wherein the step of providing comprises providing monomeric or clustered synthetic TF, or a combination thereof.

26. A method for isolating antibodies or antigen-binding molecules comprising the steps of:
providing a solution comprising antibodies or antigen-binding molecules;
contacting the solution with an affinity matrix, which affinity matrix comprises carbohydrate- or glycopepetide-based antigens that are capable of binding to said antibodies or antigen-binding molecules; and
eluting the antibodies or antigen-binding molecules from the affinity matrix.

27. The method of claim 26, wherein the step of providing comprises providing blood fluids from a patient.

28. The method of claim 27, wherein providing blood fluids from a patient further comprises immunizing a subject with a carbohydrate- or glycopeptide-based antigen and collecting blood fluids from the subject.

29. The method of claim 26, after the step of contacting, further comprising a step of washing the affinity matrix to remove unbound substrates.

30. The method of claim 26, further comprising quantifying the isolated antibodies or antigen-binding molecules.

31. The method of claim 26, further comprising characterizing the specific isolated antibodies or antigen-binding molecules.

32. The method of claim 26, wherein the antigen comprises monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

33. The method of claim 26, wherein the tumor-associated carbohydrate- or glycopeptide-based antigen is monomeric or clustered globo-H oligosaccharide, or a combination thereof.

34. The method of claim 26, wherein the tumor-associated carbohydrate- or glycopeptide-based antigen is monomeric or clustered Lewis Y oligosaccharide, or a combination thereof.

35. The method of claim 26, wherein the tumor-associated carobhydrate- or glycopetpide-based antigen is monomeric or clustered Tn, or a combination thereof.

36. The method of claim 26, wherein the tumor-associated carbohydrate- or glycopeptide-based antigen is monomeric or clustered TF, or a combination thereof.

37. The method of claim 26, wherein the antibodies or antigen-binding molecules retain their functionality.

38. A method of detecting a cancer in a subject comprising the steps of:
(a) providing a solution comprising blood fluids from a subject;
(b) contacting the solution with an affinity matrix, wherein said affinity matrix comprises tumor-associated carbohydrate- or glycopeptide-based antigens bound to the matrix;
(c) treating the affinity matrix with a reagent suitable to elute antibodies or antigen-binding molecules bound to the tumor-associated carbohydrate- or glycopeptide-based antigens present in the affinity matrix; and
(d) determining the presence of antibodies or antigen-binding molecules.

39. The method of claim 38, wherein providing blood fluids from a patient further comprises immunizing a subject with a carbohydrate- or glycopeptide-based antigen and collecting blood fluids from the subject.

40. The method of claim 38, after the step of contacting, further comprising a step of washing the affinity matrix to remove unbound substrates.

41. The method of claim 38, wherein the tumor-associated carbohydrate- or glycopeptide-based antigen comprises monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

42. The method of claim 38, further comprising repeating steps (a)-(d) at one or more specific time intervals to monitor the progress of treatment over a specific period of time of a type of cancer having a tumor-associated carbohydrate- or glycopeptide-based antigen associated therewith.

43. A method of treating cancer in a subject comprising the steps of:
(a) isolating antibodies or antigen-binding molecules, wherein the step of isolating comprises:
providing a solution comprising blood fluids from a subject;
contacting the solution with an affinity matrix, whereby said affinity matrix comprises tumor-associated carbohydrate- or glycopeptide-based antigens; and
treating the affinity matrix with a reagent suitable to elute antibodies or antigen-binding molecules bound to the tumor-associated carbohydrate- or glycopeptide-based antigens present in the affinity matrix;
(b) conjugating one or more therapeutic agents to the isolated antibodies or antigen-binding molecules; and
(c) re-administering the conjugated antibodies or antigen-binding molecules to the subject.

44. The method of claim 43, wherein the cancer is prostrate, breast, colon, ovarian, pancreatic, melanoma, neuroblastoma, or small cell lung cancer.

45. The method of claim 43, wherein the one or more therapeutic agents are radioactive isotopes or anti-cancer agents.

46. The method of claim 43, wherein the isolated antibodies are antibodies capable of binding to monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

47. The method of claim 43, wherein the antibodies are capable of binding to monomeric or clustered globo-H antigen, or a combination thereof.

48. The method of claim 43, wherein the antibodies are capable of binding to monomeric or clustered LewisY antigen, or a combination thereof.

49. The method of claim 43, wherein the antibodies are capable of binding to monomeric or clustered Tn antigen, or a combination thereof.

50. The method of claim 43, wherein the antibodies are capable of binding to monomeric or clustered TF antigen, or a combination thereof.

51. The method of claim 43, wherein said antibodies or antigen-binding molecules are naturally occurring antibodies or antigen-binding molecules.

52. The method of claim 43, wherein said antibodies are induced by a monomeric or clustered globo-H vaccine, or a combination thereof.

53. The method of claim 43, wherein said antibodies are induced by a monomeric or clustered Lewis Y vaccine, or a combination thereof.

54. The method of claim 43, wherein said antibodies are induced by a monomeric or clustered Tn vaccine, or a combination thereof.

55. The method of claim 43, wherein said antibodies are induced by a monomeric or clustered TF vaccine, or a combination thereof.

56. A method of imaging cancer metastases in a subject comprising the steps of:
(a) isolating antibodies or antigen-binding molecules, wherein the step of isolating comprises:
providing a solution comprising blood fluids from a subject;
contacting the solution with an affinity matrix, whereby said affinity matrix comprises tumor-associated carbohydrate- or glycopeptide-based antigen; and
treating the affinity matrix with a reagent suitable to elute antibodies or antigen-binding molecules bound to the tumor-associated carbohydrate- or glycopeptide-based antigens present in the affinity matrix;
(b) labeling the isolated antibodies or antigen-binding molecules with imaging agents; and
(c) re-administering the labeled antibodies or antigen-binding molecules to the subject.

57. The method of claim 56 wherein said imaging substance is a radioactive isotope.

58. The method of claim 56, wherein the isolated antibodies are antibodies capable of binding to monomeric or clustered globo-H-oligosaccharide, Lewis Y oligosaccharide, GM2, GD2, GD3, fucosyl GM1, S-Tn, Tn, TF, KH-1, N3, glycosylated segments of muc 1 and muc 2, or combinations thereof.

59. The method of claim 56, wherein the antibodies are capable of binding to monomeric or clustered globo-H antigen, or a combination thereof.

60. The method of claim 56 wherein the antibodies are capable of binding to monomeric or clustered LewisY antigen, or a combination thereof.

61. The method of claim 56, wherein the antibodies are capable of binding to monomeric or clustered Tn antigen, or a combination thereof.

62. The method of claim 56, wherein the antibodies are capable of binding to monomeric or clustered TF antigen, or a combination thereof.
